(19) United States
(12) Patent Application Publication
(10) Pub. No.: US 2012/0094979 A1
(43) Pub. Date: Apr. 19, 2012

(54) THIAZOLE OR THIADIZALOE DERIVATIVES FOR USE AS SPHINGOSINE 1-PHOSPHATE 1 (S1P1) RECEPTOR AGONISTS

(75) Inventors: James Matthew Bailey, Stevenage (GB); Emmanuel Hubert Demont, Stevenage (GB); Xichen Lin, Shanghai (CN); Feng Ren, Shanghai (CN); Christian Alan Paul Smethurst, Harlow (GB)

(73) Assignee: Glaxo Wellcome House, Greenford (GB)

(21) Appl. No.: 13/378,809

(22) PCT Filed: Jun. 17, 2010
(86) PCT No.: PCT/CN10/00868

§ 371 Date: Dec. 16, 2011

(30) Foreign Application Priority Data
Jun. 19, 2009 (GB) 0910691.5

(51) Int. Cl. A61K 31/55; A61K 31/4725; A61K 31/433; A61P 25/00; A61P 37/06; A61P 29/00; A61P 11/06; A61P 19/02; A61P 1/00; A61P 17/06; A61P 9/10; A61P 35/00; A61P 35/04; A61P 9/00; A61P 31/12; A61P 3/10; C07D 417/10

(52) U.S. Cl. 514/217.01; 540/594; 546/145; 514/307; 548/136; 514/363

(57) ABSTRACT

Thiazole or thiadizaloe derivatives of formula (I) or pharmaceutical salts thereof having pharmacological activity, processes for their preparation, pharmaceutical compositions containing them and their uses in the treatment of various disorders mediated by S1P1 receptor are disclosed.

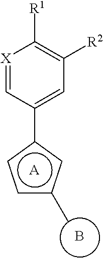

The present invention relates to novel compounds having pharmacological activity, processes for their preparation, pharmaceutical compositions containing them and their use in the treatment of various disorders.

Sphingosine 1-phosphate (S1P) is a bioactive lipid mediator formed by the phosphorylation of sphingosine by sphingosine kinases and is found in high levels in the blood. It is produced and secreted by a number of cell types, including those of hematopoietic origin such as platelets and mast cells (Okamoto et al 1998 J Biol Chem 273(42):27104; Sanchez and Hla 2004, J Cell Biochem 92:913). It has a wide range of biological actions, including regulation of cell proliferation, differentiation, motility, vascularisation, and activation of inflammatory cells and platelets (Pyne and Pyne 2000, Biochem J. 349: 385). Five subtypes of SIP responsive receptor have been described, S1P1 (Edg-1), S1P2 (Edg-5), S1P3 (Edg-3), S1P4 (Edg-6), and S1P5 (Edg-8), forming part of the G-protein coupled endothelial differentiation gene family of receptors (Chun et al 2002 Pharmacological Reviews 54:265, Sanchez and Hla 2004 J Cellular Biochemistry, 92:913). These 5 receptors show differential mRNA expression, with S1P1-3 being widely expressed, S1P4 expressed on lymphoid and hematopoietic tissues and S1P5 primarily in brain and to a lower degree in spleen. They signal via different subsets of G proteins to promote a variety of biological responses (Kluk and Hla 2002 Biochem et Biophysica Acta 1582:72, Sanchez and Hla 2004, J Cellular Biochem 92:913).

Proposed roles for the S1P1 receptor include lymphocyte trafficking, cytokine induction/suppression and effects on endothelial cells (Rosen and Goetzl 2005 Nat Rev Immunol. 5:560). Agonists of the S1P1 receptor have been used in a number of autoimmune and transplantation animal models, including Experimental Autoimmune Encephalomelitis (EAE) models of MS, to reduce the severity of the induced disease (Brinkman et al 2003 JBC 277:21453; Fujino et al 2003 J Pharmacol Exp Ther 305:70; Webb et al 2004 J Neuroimmunol 153:108; Rausch et al 2004 J Magn Reson Imaging 20:16). This activity is reported to be mediated by the effect of S1P1 agonists on lymphocyte circulation through the lymph system. Treatment with S1P1 agonists results in the sequestration of lymphocytes within secondary lymphoid organs such as the lymph nodes, inducing a reversible peripheral lymphopoenia in animal models (Chiba et al 1998, J Immunology 160:5037, Forrest et al 2004 J Pharmacol Exp Ther 309:758; Sanna et al 2004 JBC 279:13839). Published data on agonists suggests that compound treatment induces loss of the S1P1 receptor from the cell surface via internalisation (Graler and Goetzl 2004 FASEB J 18:551; Matloubian et al 2004 Nature 427:355; Jo et al 2005 Chem Biol 12:703) and it is this reduction of S1P1 receptor on immune cells which contributes to the reduction of movement of T cells from the lymph nodes back into the blood stream.

S1P1 gene deletion causes embryonic lethality. Experiments to examine the role of the S1P1 receptor in lymphocyte migration and trafficking have included the adoptive transfer of labelled S1P1 deficient T cells into irradiated wild type mice. These cells showed a reduced egress from secondary lymphoid organs (Matloubian et al 2004 Nature 427:355).

S1P1 has also been ascribed a role in endothelial cell junction modulation (Allende et al 2003 102:3665, Blood Singelton et al 2005 FASEB J 19:1646). With respect to this endothelial action, S1P1 agonists have been reported to have an effect on isolated lymph nodes which may be contributing to a role in modulating immune disorders. S1P1 agonists caused a closing of the endothelial stromal ‘gates’ of lymphatic sinuses which drain the lymph nodes and prevent lymphocyte egress (Wei wt al 2005, Nat. Immunology 6:1228).

The immunosuppressive compound FTY720 (JP11080026-A) has been shown to reduce circulating lymphocytes in animals and man, have disease modulating activity in animal models of immune disorders and reduce remission rates in relapsing remitting Multiple Sclerosis (Brinkman et al 2002 JBC 277:21453, Mandala et al 2002 Science 296:346, Fujino et al 2003 J Pharmacology and Experimental Therapeutics 305:45658, Brinkman et al 2004 American J Transplantation 4:1019, Webb et al 2004 J Neuroimmunology 153:108, Morris et al 2005 EurJ Immunol 35:3570, Chiba 2005 Pharmacology and Therapeutics 108:308, Kahan et al 2003, Transplantation 76:1079, Kappos et al 2006 New Eng J Medicine 335:1124). This compound is a prodrug that is phosphorylated in vivo by sphingosine kinases to give a molecule that has agonist activity at the S1P1, S1P3, S1P4 and S1P5 receptors. Clinical studies have demonstrated that treatment with FTY720 results in bradycardia in the first 24 hours of treatment (Kappos et al 2006 New Eng J Medicine 335:1124). The bradycardia is thought to be due to agonism at the S1P3 receptor, based on a number of cell based and animal experiments. These include the use of S1P3 knock-out animals which, unlike wild type mice, do not demonstrate bradycardia following FTY720 administration and the use of S1P1 selective compounds. (Hale et al 2004 Bioorganic & Medicinal Chemistry Letters 14:3501, Sanna et al 2004 JBC 279:13839, Koyrakh et al 2005 American J Transplantation 5:529)

Hence, there is a need for S1P1 receptor agonist compounds with selectivity over S1P3 which might be expected to show a reduced tendency to induce bradycardia.

The following patent applications describe oxadiazole derivatives as S1P1 agonists: WO03/105771, WO05/058848, WO06/047195, WO06/100633, WO06/115188, WO06/131336, WO07/024,922 and WO07/116,866.

The following patent applications describe tetrahydroisoquinolinyl-oxadiazole derivatives as S1P receptor agonists: WO06/064757, WO06/001463, WO04/113330.

WO08/064,377 describes benzocycloheptyl analogs having S1P1 receptor activity.

A structurally novel class of compounds has now been found which provides agonists of the S1P1 receptor.

The present invention provides compounds of formula (I) or a pharmaceutically acceptable salt thereof thereof:

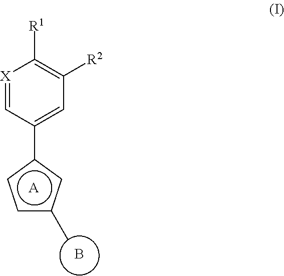

### X is CH or N;

R¹ is OR³, NHR⁴, R⁵, NR⁶R⁷, R⁸ or optionally fluorinated C₍₃₋₆₎cycloalkyl;
R² is hydrogen, halogen, cyano, trifluoromethyl, C₍₁₋₂₎alkoxy and C₍₁₋₃₎alkyl optionally substituted by halogen;
R³ and R⁴ are C₍₁₋₅₎alkyl optionally interrupted by 0 and optionally substituted by F or (CH₂)₍₀₋₁₎C₍₃₋₅₎cycloalkyl optionally substituted by F;
R⁵ is C₍₁₋₅₎alkyl optionally substituted by F;
R⁶ and R⁷ are independently selected from C₍₁₋₅₎alkyl optionally interrupted by 0 and optionally substituted by F and optionally fluorinated C₍₃₋₅₎cycloalkyl with the proviso that the combined number of carbon atoms in R⁶ and R⁷ does not exceed 6;
R⁸ is a 3 to 6 membered, nitrogen-containing heterocyclyl ring optionally substituted by F selected from aziridinyl, azetidinyl, pyrrolidinyl, piperidinyl and morpholinyl, all attached via the nitrogen atom;

A is a 5-membered heterocyclic ring selected from the following:

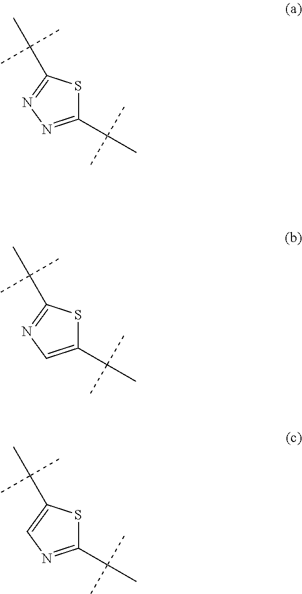

B is a bicyclic ring selected from the following:

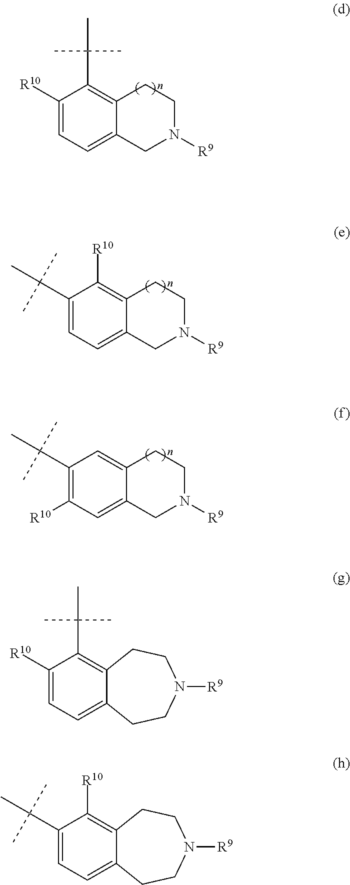

R⁹ is hydrogen or (CH₂)₁₋₄O₂H;
R¹⁰ is hydrogen or C₍₁₋₃₎alkyl optionally substituted by halogen; and
n is 0, 1 or 2.

In one embodiment X is N. In another embodiment X is CH.

In one embodiment R¹ is OR³ or R5.

In one embodiment R³ is isopropyl.

In one embodiment R⁵ is butyl.

In one embodiment R² is chloro or cyano.

In one embodiment A is (a). In another embodiment A is (b).

In a further embodiment A is (c).

In one embodiment B is (e), (d) or (h). In another embodiment B is (e).

In one embodiment R⁹ is hydrogen or (CH₂)₂₋₃CO₂H

In one embodiment R¹⁰ is hydrogen, methyl or ethyl.

In one embodiment n is 0 or 1.

In one embodiment

### X is N or CH;

### R¹ is OR³ or R⁵;

R³ is isopropyl;
R⁵ is butyl;
R² is chloro or cyano;

### A is (a) or (b);

### B is (e), (d) or (h);

R⁹ is hydrogen or (CH₂)₂₋₃CO₂H;
R¹⁰ is hydrogen, methyl or ethyl; and
n is 0 or 1.

In one embodiment

### X is N or CH;

### R¹ is OR³;

R³ is isopropyl;
R² is chloro or cyano;

### A is (a), (b) or (c);

### B is (e), (d) or (h);

R⁹ is hydrogen or (CH₂)₂₋₃CO₂H;
R¹⁰ is hydrogen or methyl; and
n is 0 or 1.

In one embodiment

### X is N or CH;

### R¹ is OR³;

R³ is isopropyl;
R² is cyano;

### A is (a);

### B is (e) or (h);

R⁹ is hydrogen or (CH₂)₂CO₂H;
R¹⁰ is hydrogen or methyl; and
n is 1.

The term “alkyl” as a group or part of a group e.g. alkoxy or hydroxyalkyl refers to a straight or branched alkyl group in all isomeric forms. The term “C₍₁₋₆₎alkyl” refers to an alkyl group, as defined above, containing at least 1, and at most 6 carbon atoms Examples of such alkyl groups include methyl, ethyl, propyl, iso-propyl, n-butyl, iso-butyl, sec-butyl, or tert-butyl. Examples of such alkoxy groups include methoxy, ethoxy, propoxy, iso-propoxy, butoxy, iso-butoxy, sec-butoxy and tert-butoxy.

Suitable C₍₃₋₆₎cycloalkyl groups include cyclopropyl, cyclobutyl, cyclopentyl and cyclohexyl.

As used herein, the term “halogen” refers to fluorine (F), chlorine (Cl), bromine (Br), or iodine (I) and the term “halo” refers to the halogen: fluoro (—F), chloro (—Cl), bromo (—Br) and iodo (—I).

The term “substituted” includes the implicit provision that substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. In certain embodiments, alkyl groups optionally substituted by F or OH may be multiply substituted on multiple carbon atoms.

In certain of the compounds of formula (I), dependent upon the nature of the substituent there are chiral carbon atoms and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

It is understood that certain compounds of the invention contain both acidic and basic groups and may therefore exist as zwitterions at certain pH values.

Suitable compounds of the invention are:
7-(2-{3-Chloro-4-[(1-methylethyl)oxy]phenyl}-1,3-thiazol-5-yl)-2,3,4,5-tetrahydro-1H-3-benzazepine
7-(5-{3-Chloro-4-[(1-methylethyl)oxy]phenyl}-1,3-thiazol-2-yl)-2,3,4,5-tetrahydro-1H-3-benzazepine
4-[7-(2-{3-Chloro-4-[(1-methylethyl)oxy]phenyl}-1,3-thiazol-5-yl)-1,2,4,5-tetrahydro-3H-3-benzazepin-3-yl]butanoic acid
4-[7-(5-{3-Chloro-4-[(1-methylethyl)oxy]phenyl}-1,3-thiazol-2-yl)-1,2,4,5-tetrahydro-3H-3-benzazepin-3-yl]butanoic acid
7-(5-{3-Chloro-4-[(1-methylethyl)oxy]phenyl}-1,3,4-thiadiazol-2-yl)-2,3,4,5-tetrahydro-1H-3-benzazepine
2-[(1-Methylethyl)oxy]-5-[5-(2,3,4,5-tetrahydro-1H-3-benzazepin-7-yl)-1,3,4-thiadiazol-2-yl]benzonitrile trifluoroacetic acid
4-[7-(5-{3-Cyano-4-[(1-methylethyl)oxy]phenyl}-1,3,4-thiadiazol-2-yl)-1,2,4,5-tetrahydro-3H-3-benzazepin-3-yl]butanoic acid hydrochloride
2-[(1-Methylethyl)oxy]-5-[5-(5-methyl-1,2,3,4-tetrahydro-6-isoquinolinyl)-1,3,4-thiadiazol-2-yl]benzonitrile
3-[6-(5-{3-Cyano-4-[(1-methylethyl)oxy]phenyl}-1,3,4-thiadiazol-2-yl)-5-methyl-3,4-dihydro-2(1H)-isoquinolinyl]propanoic acid
4-[6-(5-{3-Cyano-4-[(1-methylethyl)oxy]phenyl}-1,3,4-thiadiazol-2-yl)-5-methyl-3,4-dihydro-2(1H)-isoquinolinyl]butanoic acid
2-[(1-Methylethyl)oxy]-5-[5-(1,2,3,4-tetrahydro-6-isoquinolinyl)-1,3,4-thiadiazol-2-yl]benzonitrile
2-[(1-Methylethyl)oxy]-5-[5-(1,2,3,4-tetrahydro-5-isoquinolinyl)-1,3,4-thiadiazol-2-yl]benzonitrile
5-(5-{3-Chloro-4-[(1-methylethyl)oxy]phenyl}-1,3,4-thiadiazol-2-yl)-2,3-dihydro-1H-isoindole trifluoroacetic acid
3-[5-(5-{3-Chloro-4-[(1-methylethyl)oxy]phenyl}-1,3,4-thiadiazol-2-yl)-1,3-dihydro-2H-isoindol-2-yl]propanoic acid
3-[6-(2-{3-cyano-4-[(1-methylethyl)oxy]phenyl}-1,3-thiazol-5-yl)-5-ethyl-3,4-dihydro-2(1H)-isoquinolinyl]propanoic acid
4-[6-(2-{3-cyano-4-[(1-methylethyl)oxy]phenyl}-1,3-thiazol-5-yl)-5-ethyl-3,4-dihydro-2(1H)-isoquinolinyl]butanoic acid
3-[6-(5-{3-chloro-4-[(1-methylethyl)oxy]phenyl}-1,3,4-thiadiazol-2-yl)-5-ethyl-3,4-dihydro-2(1H)-isoquinolinyl]propanoic acid
4-[6-(5-{3-chloro-4-[(1-methylethyl)oxy]phenyl}-1,3,4-thiadiazol-2-yl)-5-ethyl-3,4-dihydro-2(1H)-isoquinolinyl]butanoic acid
3-[6-{5-[3-cyano-4-(2-methylpropyl)phenyl]-1,3,4-thiadiazol-2-yl}-5-ethyl-3,4-dihydro-2(1H)-isoquinolinyl]propanoic acid
4-[6-(2-{3-cyano-4-[(1-methylethyl)oxy]phenyl}-1,3-thiazol-5-yl)-5-ethyl-3,4-dihydro-2(1H)-isoquinolinyl]butanoic acid
   or pharmaceutically acceptable salts thereof.

Pharmaceutically acceptable derivatives of compounds of formula (I) include any pharmaceutically acceptable salt, ester or salt of such ester of a compound of formula (I) which, upon administration to the recipient is capable of providing (directly or indirectly) a compound of formula (I) or an active metabolite or residue thereof.

The compounds of formula (I) can form salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms. Salts may also be prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

Pharmaceutically acceptable acid addition salts may be prepared conventionally by reaction with the appropriate acid or acid derivative. Pharmaceutically acceptable salts with bases may be prepared conventionally by reaction with the appropriate inorganic or organic base.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Included within the scope of the invention are all salts, solvates, hydrates, complexes, polymorphs, prodrugs, radiolabelled derivatives, stereoisomers and optical isomers of the compounds of formula (I).

The potencies and efficacies of the compounds of this invention for the S1P1 receptor can be determined by GTPγS assay performed on the human cloned receptor as described herein. Compounds of formula (I) have demonstrated agonist activity at the S1P1 receptor, using functional assays described herein.

Compounds of formula (I) and their pharmaceutically acceptable salts are therefore of use in the treatment of conditions or disorders which are mediated via the S1P1 receptor. In particular the compounds of formula (I) and their pharmaceutically acceptable salts are of use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are therefore of use in the treatment of lupus erythematosis.

Compounds of formula (I) and their pharmaceutically acceptable salts are therefore of use in the treatment of psoriasis.

Compounds of formula (I) and their pharmaceutically acceptable salts are therefore of use in the treatment of multiple sclerosis.

Compounds of formula (I) and their pharmaceutically acceptable salts may also be of use in the treatment of Parkinson's Disease, Alzheimer's disease, Huntington's chorea, amyotrophic lateral sclerosis, spinal muscular atrophy, polyglutamine expansion disorders, vascular dementia, Down's syndrome, HIV dementia, dementia, ocular diseases including glaucoma, aged related macular degeneration, cataracts, traumatic eye injury, diabetic retinopathy, traumatic brain injury, stroke, tauopathies and hearing loss.

It is to be understood that “treatment” as used herein includes prophylaxis as well as alleviation of established symptoms.

Thus the invention also provides compounds of formula (I) or pharmaceutically acceptable salts thereof, for use as therapeutic substances, in particular in the treatment of the conditions or disorders mediated via the S1P1 receptor. In particular the invention provides a compound of formula (I) or a pharmaceutically acceptable salt thereof for use as a therapeutic substance in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are of use as therapeutic substances in the treatment of lupus erythematosis.

Compounds of formula (I) and their pharmaceutically acceptable salts are of use as therapeutic substances in the treatment of psoriasis.

Compounds of formula (I) and their pharmaceutically acceptable salts are of use as therapeutic substances in the treatment of multiple sclerosis.

The invention further provides a method of treatment of conditions or disorders in mammals including humans which can be mediated via the S1P1 receptor, which comprises administering to the sufferer a therapeutically safe and effective amount of a compound of formula (I) or a pharmaceutically acceptable salt thereof. In particular the invention provides a method of treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes, which comprises administering to the sufferer a therapeutically safe and effective amount of a compound of formula (I) or a pharmaceutically acceptable salt thereof.

The invention provides a method of treatment of lupus erythematosis, which comprises administering to the sufferer a therapeutically safe and effective amount of a compound of formula (I) or a pharmaceutically acceptable salt thereof.

The invention provides a method of treatment of psoriasis, which comprises administering to the sufferer a therapeutically safe and effective amount of a compound of formula (I) or a pharmaceutically acceptable salt thereof.

The invention provides a method of treatment of multiple sclerosis, which comprises administering to the sufferer a therapeutically safe and effective amount of a compound of formula (I) or a pharmaceutically acceptable salt thereof.

In another aspect, the invention provides for the use of a compound of formula (I) or a pharmaceutically acceptable salt thereof in the manufacture of a medicament for use in the treatment of the conditions or disorders mediated via the S1P1 receptor.

In particular the invention provides a compound of formula (I) or a pharmaceutically acceptable salt thereof for use in the manufacture of a medicament for use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are of use in the manufacture of a medicament for use in the treatment of lupus erythematosis.

Compounds of formula (I) and their pharmaceutically acceptable salts are of use in the manufacture of a medicament for use in the treatment of psoriasis.

Compounds of formula (I) and their pharmaceutically acceptable salts are of use in the manufacture of a medicament for use in the treatment of multiple sclerosis.

In order to use the compounds of formula (I) and pharmaceutically acceptable salts thereof in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salts thereof and a sterile vehicle. Formulations for injection may be presented in unit dosage form e.g. in ampoules or in multi-dose, utilising a compound of the invention or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts thereof may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of formula (I) or pharmaceutically acceptable salts thereof, may be formulated as solutions for administration via a suitable metered or unitary dose device or alternatively as a powder mix with a suitable carrier for administration using a suitable delivery device. Thus compounds of formula (I) or pharmaceutically acceptable salts thereof may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, 1.0 to 500 mg or 1.0 to 200 mg and such unit doses may be administered more than once a day, for example two or three times a day.

Compounds of formula (I) or pharmaceutically acceptable salts thereof may be used in combination preparations, in combination with other active ingredients. For example, the compounds of the invention may be used in combination with cyclosporin A, methotrexate, steriods, rapamycin, proinflammatory cytokine inhibitors, immunomodulators including biologicals or other therapeutically active compounds.

The subject invention also includes isotopically-labeled compounds, which are identical to those recited in formulas I and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable saltss of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula (I) and following of this invention can generally be prepared by carrying out the procedures disclosed in the Schemes and/or in the Examples below, by substituting a readily available isotopically labelled reagent for a non-isotopically labeled reagent.

In a further aspect, this invention provides processes for preparation of a compound of formula (I).

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

### ABBREVIATIONS

g—grams
mg—milligrams
ml—millilitres
ul—microlitres
BOC₂O—bis(1,1-dimethylethyl)dicarbonate
MeCN—acetonitrile
MeOH—methanol
EtOH—ethanol
Et2O—diethyl ether
EtOAc—ethyl acetate
DCM—dichloromethane
DIAD—diisopropyl azodicarboxylate
DIPEA—diisopropylethylamine
DME—1,2-bis(methyloxy)ethane

### DMF—N,N-dimethylformamide

DMSO—dimethylsulphoxide
d₆DMSO—deuterated dimethylsulphoxide
EDAC—N-(3-Dimethylaminopropyl)-N′-ethylcarbodiimide hydrochloride
EDC—N-(3-Dimethylaminopropyl)-N′-ethylcarbodiimide hydrochloride
EDCI—N-(3-Dimethylaminopropyl)-N′-ethylcarbodiimide hydrochloride

### HOBT/HOBt—Hydroxybenzotriazole

IPA—isopropylalcohol
MeOD—deuterated methanol

### NCS—N-chlorosuccinimide

### PPh₃—Triphenylphosphine

PyBOP—Benzotriazol-1-yl-oxytripyrrolidinophosphonium hexafluorophosphate
THF—tetrahydrofuran
TFA—trifluoroacetic acid
dba—dibenzylidene acetone
RT—room temperature
° C. —degrees Celsius

### M—Molar

H—proton
s—singlet
d—doublet
t—triplet
q—quartet
MHz—megahertz
MeOD—deuterated methanol

### LCMS—Liquid Chromatography Mass Spectrometry

### LC/MS—Liquid Chromatography Mass Spectrometry

MS—mass spectrometry

### ES—Electrospray

MH+—mass ion+H+
MDAP—mass directed automated preparative liquid chromatography.
sat. —saturated
SCX—solid phase cation exchange chromatography

### General Chemistry Section

The methods described below are given for illustrative purposes, intermediates in the preparation of the examples may not necessarily have been prepared from the specific batches described.

### Preparation 1

### 2-{3-Chloro-4-[(1-methylethyl)oxy]phenyl}-1,3-thiazole

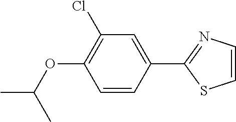

A flask was charged with 2-bromothiazole (CAS#3034-53-5, commercially available from Aldrich, 492 mg, 3.00 mmol), {3-chloro-4-[(1-methylethyl)oxy]phenyl}boronic acid (CAS#480438-56-0, commercially available from Aldrich, 772 mg, 3.60 mmol), a 2M Na₂CO₃ aqueous solution (3.00 ml, 6.00 mmol) and Ph₃P (79 mg, 0.300 mmol) then filled with EtOH (1 ml) and toluene (7 ml). The resulting mixture was de-gassed several times under vacuum (ca 15 mbar) then flushed with nitrogen. Palladium(II)acetate (20.20 mg, 0.090 mmol) was then added and the procedure of de-gassing repeated. The resulting mixture was stirred 5 hours under nitrogen at 100° C., at room temperature for 16 h then concentrated in vacuo. The residue was partitioned between AcOEt and water and the layers were separated. The organic phase was washed with water and brine, dried over MgSO₄ and concentrated in vacuo. Purification of the residue by flash chromatography on silica gel (c-Hexane/AcOEt: 0 to 10% gradient) gave 2-{3-chloro-4-[(1-methylethyl)oxy]phenyl}-1,3-thiazole (454 mg, 59%) as a yellow oil.

LCMS: retention time 1.32 min; [M+H]⁺=254.1

### Preparation 2

### 5-Bromo-2-{3-chloro-4-[(1-methylethyl)oxy]phenyl}-1,3-thiazole

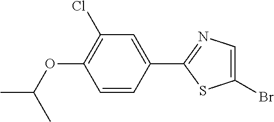

2-{3-Chloro-4-[(1-methylethyl)oxy]phenyl}-1,3-thiazole (Preparation 1) (446 mg, 1.758 mmol) in CHCl₃ (5 ml) at room temperature was treated with a solution of bromine (0.100 ml, 1.933 mmol) in CHCl₃ (2 ml). After 20 minutes, a further quantity of bromine, (45μl, 0.879 mmol) was added and the resulting mixture was stirred at room temperature for 16 h. A further quantity of bromine, (45μl, 0.879 mmol) was added and the resulting mixture was stirred for 2 hours then concentrated in vacuo. The orange residue formed was triturated with Et₂O and the precipitate formed was filtered off, and washed with Et₂O then purified by flash chromatography on silica gel (c-Hexane/AcOEt: 0 to 10%) to give 5-bromo-2-{3-chloro-4-[(1-methylethyl)oxy]phenyl}-1,3-thiazole (438 mgs, 74%) as a colourless solid.

LCMS: Retention time 1.55 min, [M+H]⁺=334.0 (1 Br)

### Preparation 3

### 1,1-Dimethylethyl 7-(4,4,5,5-tetramethyl-1,3,2-dioxaborolan-2-yl)-1,2,4,5-tetrahydro-3H-3-benzazepine-3-carboxylate

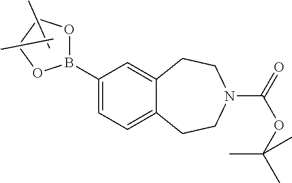

1,1-Dimethylethyl 7-{[(trifluoromethyl)sulfonyl]oxy}-1,2,4,5-tetrahydro-3H-3-benzazepine-3-carboxylate (may be prepared as described in WO2002040471, 22 g, 55.7 mmol) and 4,4,4′,4′,5,5,5′,5′-octamethyl-2,2′-bi-1,3,2-dioxaborolane (15.55 g, 61.25 mmol) were dissolved in 1,4-dioxane (250 ml) and the mixture was de-gassed 15 minutes under vacuum (ca 15 mbar) then quenched with nitrogen. 1,1′-bis(diphenylphosphino)ferrocenedichloro palladium(II) (2.92 g, 3.34 mmol) and 1,1′-bis(diphenylphosphino)ferrocene (1.85 g, 3.34 mmol) were added and the resulting mixture was stirred 10 minutes under nitrogen before potassium acetate (16.39 g, 167.0 mmol) was added. The resulting mixture was stirred at 80° C. for 3 hours then cooled to room temperature and diluted with AcOEt, water and brine. The two layers were separated and the organic phase dried over Na₂SO₄ and concentrated in vacuum. Purification of the residue by flash chromatography on silica gel (c-Hexane/AcOEt: 10%) gave 1,1-dimethylethyl 7-(4,4,5,5-tetramethyl-1,3,2-dioxaborolan-2-yl)-1,2,4,5-tetrahydro-3H-3-benzazepine-3-carboxylate (4.5 g, 22%) as a white solid.

### Preparation 4

### 1,1-Dimethylethyl 7-(2-{3-chloro-4-[(1-methylethyl)oxy]phenyl}-1,3-thiazol-5-yl)-1,2,4,5-tetrahydro-3H-3-benzazepine-3-carboxylate

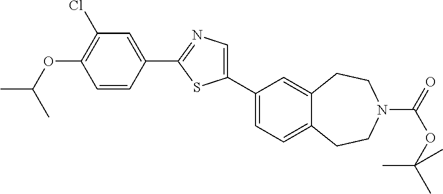

A mixture of 1,1-dimethylethyl 7-(4,4,5,5-tetramethyl-1,3,2-dioxaborolan-2-yl)-1,2,4,5-tetrahydro-3H-3-benzazepine-3-carboxylate (Preparation 3) (532 mg, 1.425 mmol) and 5-bromo-2-{3-chloro-4-[(1-methylethyl)oxy]phenyl}-1,3-thiazole (Preparation 2) (395 mg, 1.187 mmol) in 1,4-dioxane (7 ml) and saturated NaHCO₃ aqueous solution (3 ml), at room temperature was de-gassed under vacuum (ca 15 mbar) and flushed several times with nitrogen. 1,1′-bis(diphenylphosphino)ferrocenedichloro palladium(II) (87 mg, 0.119 mmol) was then added and the system flushed again with nitrogen. The reaction mixture was stirred at 90° C. for 2 hours then cooled to room temperature and concentrated in vacuo. The residue was partitioned between AcOEt and water and the layers were separated. The organic phase was washed with water then brine, dried over MgSO₄ and concentrated in vacuo. Purification of the residue by flash chromatography on silica gel (c-Hexane/AcOEt: 0 to 20%) gave 1,1-dimethylethyl 7-(2-{3-chloro-4-[(1-methylethyl)oxy]phenyl}-1,3-thiazol-5-yl)-1,2,4,5-tetrahydro-3H-3-benzazepine-3-carboxylate (463 mg, 69%) as a yellow gum.

LCMS: Retention time 1.71 min, [M+H]⁺=499.3

### Preparation 5

### 1,1-Dimethylethyl 7-(1,3-thiazol-2-yl)-1,2,4,5-tetrahydro-3H-3-benzazepine-3-carboxyla

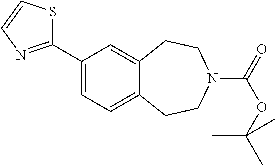

A flask was charged with 2-bromothiazole (CAS #3034-53-5, commercially available from Aldrich, 492 mg, 3.00 mmol), 1,1-dimethylethyl 7-(4,4,5,5-tetramethyl-1,3,2-dioxaborolan-2-yl)-1,2,4,5-tetrahydro-3H-3-benzazepine-3-carboxylate (Preparation 3) (1120 mg, 3.00 mmol) and Pd(Ph₃P)₄ (173 mg, 0.150 mmol) then filled with a 2M Na₂CO₃ aqueous solution (4 ml, 8.00 mmol) and DME (6 ml). The resulting mixture was stirred at 120° C. for 1 hour under microwave irradiation. Tetrakis(triphenylphosphine)palladium(0) (173 mg, 0.150 mmol) was then added and the resulting mixture was stirred at 120° C. for another 3 hours under microwave irradiation then cooled to room temperature. The mixture was diluted with AcOEt and the two layers were separated. The organic phase was washed with a 2M Na₂CO₃ aqueous solution then brine, dried over MgSO₄ and concentrated in vacuo. Purification of the residue by flash chromatography on silica gel (c-Hexane/AcOEt: 0 to 10% gradient) gave 1,1-dimethylethyl 7-(1,3-thiazol-2-yl)-1,2,4,5-tetrahydro-3H-3-benzazepine-3-carboxylate (368 mgs, 37%) as a pale yellow gum.

LCMS: retention time 1.29 min; [M+H]⁺=331.20

### Preparation 6

### 1,1-Dimethylethyl 7-(5-bromo-1,3-thiazol-2-yl)-1,2,4,5-tetrahydro-3H-3-benzazepine-3-carboxylate

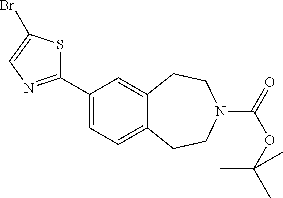

A solution of 1,1-dimethylethyl 7-(1,3-thiazol-2-yl)-1,2,4,5-tetrahydro-3H-3-benzazepine-3-carboxylate (Preparation 5) (359 mg, 1.086 mmol) in CH₃CN (5 ml) at room temperature was treated with NBS (213 mg, 1.195 mmol) and the resulting mixture was stirred at this temperature for 60 hours, then decanted from a small amount of tar and concentrated in vacuo. Purification of the residue by flash chromatography on silica gel (c-Hexane/AcOEt: 0 to 20% gradient) gave 1,1-dimethylethyl 7-(5-bromo-1,3-thiazol-2-yl)-1,2,4,5-tetrahydro-3H-3-benzazepine-3-carboxylate (307 mgs, 69%) as a colourless solid.

LCMS: retention time 1.51 min; [M+H]⁺=411.09 (1 Br)

### Preparation 7

### 1,1-Dimethylethyl 7-(5-{3-chloro-4-[(1-methylethyl)oxy]phenyl}-1,3-thiazol-2-yl)-1,2,4,5-tetrahydro-3H-3-benzazepine-3-carboxylate

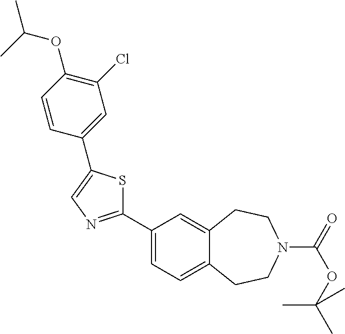

A flask was charged with 1,1-dimethylethyl 7-(5-bromo-1,3-thiazol-2-yl)-1,2,4,5-tetrahydro-3H-3-benzazepine-3-carboxylate (Preparation 6) (301 mg, 0.735 mmol), {3-chloro-4-[(1-methylethyl)oxy]phenyl}boronic acid (CAS#: 480438-56-0, commercially available from Aldrich, 189 mg, 0.882 mmol) then filled with 1,4-dioxane (5 ml) and a saturated NaHCO₃ aqueous solution (2 ml, 0.735 mmol) then de-gassed under vacuum (ca 15 mbar) and flushed with nitrogen several times. 1,1′-bis(diphenylphosphino)ferrocenedichloro palladium(II) (53.8 mg, 0.074 mmol) was then added and the procedureof de-gassing repeated. The reaction mixture was then stirred under nitrogen at 90° C. for 1 hour then cooled to room temperature and concentrated in vacuo. The residue was partitioned between AcOEt and water and the layers were separated. The organic phase was washed with water and brine, dried over MgSO₄ and concentrated in vacuo. Purification of the residue by flash chromatography on silica gel (c-hexane/AcOEt: 0 to 20% gradient) gave 1,1-dimethylethyl 7-(5-{3-chloro-4-[(1-methylethyl)oxy]phenyl}-1,3-thiazol-2-yl)-1,2,4,5-tetrahydro-3H-3-benzazepine-3-carboxylate (293 mgs, 80%) as a colourless, crystalline solid.

LCMS: retention time 1.68 min; [M+H]⁺=499.20

### Preparation 8

### Ethyl 4-[7-(2-{3-chloro-4-[(1-methylethyl)oxy]phenyl}-1,3-thiazol-5-yl)-1,2,4,5-tetrahydro-3H-3-benzazepin-3-yl]butanoate

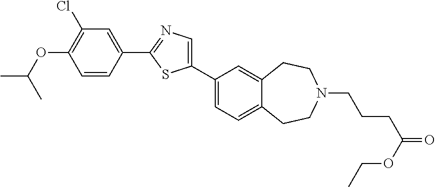

A mixture of 7-(2-{3-chloro-4-[(1-methylethyl)oxy]phenyl}-1,3-thiazol-5-yl)-2,3,4,5-tetrahydro-1H-3-benzazepine hydrochloride (Example 1) (256 mg, 0.642 mmol), ethyl 4-bromobutyrate (0.110 ml, 0.770 mmol) and K₂CO₃ (355 mg, 2.57 mmol) in dry DMF (4 ml) was heated under nitrogen at 100° C. for 3 hours then cooled to room temperature and concentrated in vacuo. The residue was partitioned between AcOEt and water and the layers were separated. The organic phase was washed with water and brine, dried over MgSO₄ and concentrated in vacuo. Purification of the residue by flash chromatography on silica gel (c-Hexane/AcOEt: 0 to 100% gradient) gave ethyl 4-[7-(2-{3-chloro-4-[(1-methylethyl)oxy]phenyl}-1,3-thiazol-5-yl)-1,2,4,5-tetrahydro-3H-3-benzazepin-3-yl]butanoate (268 mgs, 80%) as a yellow solid.

LCMS: retention time 1.15 min; [M+H]⁺=513.3

### Preparation 9

### Ethyl 4-[7-(5-{3-chloro-4-[(1-methylethyl)oxy]phenyl}-1,3-thiazol-2-O-1,2,4,5-tetrahydro-3H-3-benzazepin-3-yl]butanoate

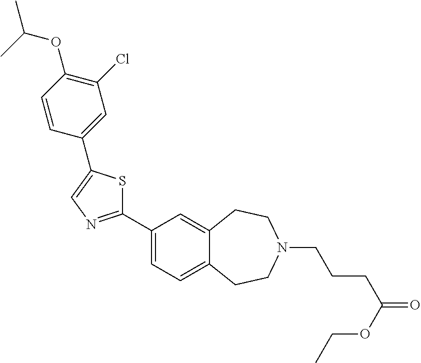

A flask was charged with 7-(5-{3-chloro-4-[(1-methylethyl)oxy]phenyl}-1,3-thiazol-2-yl)-2,3,4,5-tetrahydro-1H-3-benzazepine hydrochloride (Example 2) (207 mg, 0.519 mmol), ethyl 4-bromobutanoate (0.089 ml, 0.623 mmol) and K₂CO₃ (287 mg, 2.075 mmol) then filled with DMF (3 ml) and the resulting mixture was stirred under nitrogen at 100° C. for 1 hour then cooled to room temperature and concentrated in vacuo. The residue was partitioned between AcOEt and water and the layers were separated. The organic phase was washed with water then brine, dried over MgSO₄ and concentrated in vacuo. Purification of the residue by flash chromatography on silica gel (c-Hexane/AcOEt: 0 to 100% gradient) gave ethyl 4-[7-(5-{3-chloro-4-[(1-methylethyl)oxy]phenyl}-1,3-thiazol-2-yl)-1,2,4,5-tetrahydro-3H-3-benzazepin-3-yl]butanoate (215 mg, 80%) as a yellow gum.

LCMS: retention time 1.24 min; [M+H]⁺=513.27

### Preparation 10

### 5-(5-Amino-1,3,4-thiadiazol-2-yl)-2-[(1-methylethyl)oxy]benzonitrile

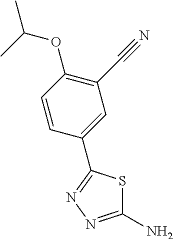

A flask was charged with 3-cyano-4-[(1-methylethyl)oxy]benzoic acid (CAS #: 258273-31-3, commercially available from Boaopharma, 20.9 g, 102 mmol) and hydrazinecarbothioamide (CAS#: 79-19-6, commercially available from Aldrich, 13.9 g, 153 mmol) then phosphorus oxychloride (CAS#: 10025-87-3, commercially available from Aldrich, 90 g, 587 mmol) was added. The resulting mixture was stirred at 90° C. for 3 hours then cooled to room temperature and added very carefully in small portions to a 5M NaOH aqueous solution cooled with an ice bath such that the temperature never rose above 35° C. The resulting mixture was basified to pH 10 (using a 5M NaOH aqueous solution) then stirred 30 minutes. The precipitate formed was collected by filtration and dissolved in DCM (1 l) and MeOH (50 ml). The organic phase was washed with water (500 ml), dried over MgSO₄ and concentrated in vacuo to give 5-(5-amino-1,3,4-thiadiazol-2-yl)-2-[(1-methylethyl)oxy]benzonitrile (26.3 g, 99% yield) as pale yellow solid which was used in the next step without further purification.

LCMS: retention time 0.86 min; [M+H]⁺=261.13

### Preparation 11

### 5-(5-Bromo-1,3,4-thiadiazol-2-yl)-2-[(1-methylethyl)oxy]benzonitrile

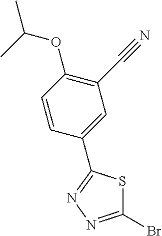

Cupric bromide (19.63 g, 88 mmol) and tert-butyl nitrite (10.44 ml, 88 mmol) were dissolved in CH₃CN (400 ml) and the resulting mixture was stirred for 10 minutes at room temperature. 5-(5-Amino-1,3,4-thiadiazol-2-yl)-2-[(1-methylethyl)oxy]benzonitrile (Preparation 10) (13 g, 40.0 mmol) was then added in small portions over 30 minutes. The resulting mixture was stirred for 1 hour at room temperature, at 70° C. for 2 hours then cooled to room temperature and concentrated in vacuo. The residue was dissolved in AcOEt (600 ml) and MeOH (50 ml) and stirred at reflux for 1 hour. The insoluble material was filtered through a silica pad and rinsed with AcOEt (2×200 ml). The combined organic phases were washed with a 1M HCl aqueous solution (300 ml), dried over MgSO₄ and concentrated in vacuo. Purification of the residue by flash chromatography on silica gel (c-Hexane/AcOEt: 0 to 100% gradient) gave 5-(5-bromo-1,3,4-thiadiazol-2-yl)-2-[(1-methylethyl)oxy]benzonitrile (8.8 g, 67.9% yield) as a pale yellow solid.

LCMS: retention time 1.13 min; [M+H]⁺=325.7 (1 Br)

### Preparation 12

### 5-{(3-Chloro-4-[(1-methylethyl)oxy]phenyl}-1,3,4-thiadiazol-2-amine

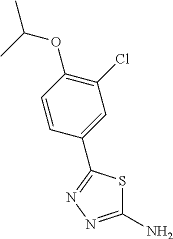

A flask was charged with 3-chloro-4-[(1-methylethyl)oxy]benzoic acid (CAS#: 213598-07-3, commercially available from Boaopharma, 25 g, 116 mmol) and hydrazinecarbothioamide (CAS#: 79-19-6, commercially available from Aldrich, 15.92 g, 175 mmol). Phosphorus oxychloride (CAS#: 10025-87-3, commercially available from Aldrich, 50 ml, 556 mmol) was then cautiously added and the resulting mixture was stirred for 20 minutes at room temperature, at 90° C. for 18 hours then cooled to room temperature. The mixture was very cautiously added dropwise to a vigorously stirred mixture of ice and water (1 l). The resulting mixture was basified (pH 12) with a 10M NaOH aqueous solution and stirred for 30 minutes while cooling with an ice/water bath. The oily sludge remaining was collected by filtration then dissolved in DCM (1 l). The organic phase was washed with brine, then dried over MgSO₄ and concentrated in vacuo to give 5-{3-chloro-4-[(1-methylethyl)oxy]phenyl}-1,3,4-thiadiazol-2-amine (9.8 g, 31.2% yield) as a brown solid which was used in the next step (Preparation 13) without further purification.

LCMS: retention time 1.02 min; [M+H]⁺=270.05

### Preparation 13

### 2-Bromo-5-{3-chloro-4-[(1-methylethyl)oxy]phenyl}-1,3,4-thiadiazole

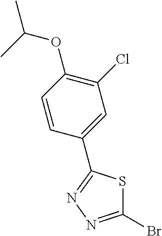

A flask was charged with cupric bromide (15.73 g, 70.4 mmol) and tert-butyl nitrite (7.26 g, 70.4 mmol) then filled with CH₃CN (300 ml). The resulting mixture was stirred at room temperature for 30 minutes, then treated with small portions of a slurry of 5-{3-chloro-4-[(1-methylethyl)oxy]phenyl}-1,3,4-thiadiazol-2-amine (Preparation 12) (9.5 g, 35.2 mmol) over 1 hour. The resulting mixture was stirred at room temperature for 1 hour, then at 60° C. for 1 hour then cooled to room temperature and concentrated in vacuo. The residue was dissolved in AcOEt (400 ml) and water (50 ml) added, giving a thick suspension which was filtered through celite. The filtrate was washed with water (400 ml) and brine (300 ml), dried over MgSO₄ and concentrated in vacuo. Purification of the residue by flash chromatography on silica gel (c-Hexane/AcOEt: 0 to 30% gradient) gave 2-bromo-5-{3-chloro-4-[(1-methylethyl)oxy]phenyl}-1,3,4-thiadiazole (4.4 g, 37%) as a yellow solid.

LCMS: retention time 1.33 min; [M+H]⁺=335.02 (1 Br).

### Preparation 14

### 1,1-Dimethylethyl 7-(5-{3-chloro-4-[(1-methylethyl)oxy]phenyl}-1,3,4-thiadiazol-2-yl)-1,2,4,5-tetrahydro-3H-3-benzazepine-3-carboxylate

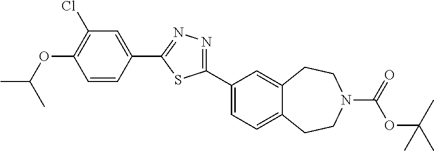

A mixture of 1,1-dimethylethyl 7-(4,4,5,5-tetramethyl-1,3,2-dioxaborolan-2-yl)-1,2,4,5-tetrahydro-3H-3-benzazepine-3-carboxylate (Preparation 3) (373 mg, 0.999 mmol) and 2-bromo-5-{3-chloro-4-[(1-methylethyl)oxy]phenyl}-1,3,4-thiadiazole (Preparation 13) (367 mg, 1.100 mmol) in 1,4-dioxane (3 ml) and a saturated NaHCO₃ aqueous solution (0.6 ml, 0.999 mmol), at room temperature was de-gassed and flushed several times with nitrogen. 1,1′-bis(diphenylphosphino)ferrocenedichloro palladium(II) (73.1 mg, 0.100 mmol) was then added and the system flushed again. The resulting mixture was stirred at 100° C. for 20 hours then cooled to room temperature and concentrated in vacuo. The residue was partitioned between AcOEt and water and the layers were separated. The organic phase was washed with water and brine, dried over MgSO₄ and concentrated in vacuo. Purification of the residue by flash chromatography on silica gel (c-Hexane/AcOEt: 0 to 20% gradient) gave 1,1-dimethylethyl 7-(5-{3-chloro-4-[(1-methylethyl)oxy]phenyl}-1,3,4-thiadiazol-2-yl)⁻1,2,4,5-tetrahydro-3H-3-benzazepine-3-carboxylate (100 mg, 20%) as an orange foam.

LCMS: retention time 1.62 min; [M+H]⁺=500.3

### Preparation 15

### 1,1-Dimethylethyl 7-(5-{3-cyano-4-[(1-methylethyl)oxy]phenyl}-1,3,4-thiadiazol-2-yl)-1,2,4,5-tetrahydro-3H-3-benzazepine-3-carboxylate

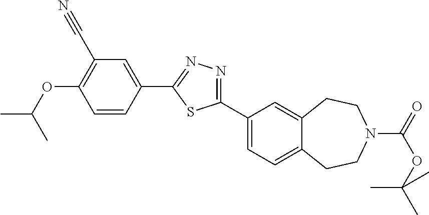

A mixture of 1,1-dimethylethyl 7-(4,4,5,5-tetramethyl-1,3,2-dioxaborolan-2-yl)-1,2,4,5-tetrahydro-3H-3-benzazepine-3-carboxylate (Preparation 3) (203 mg, 0.544 mmol), 5-(5-bromo-1,3,4-thiadiazol-2-yl)-2-[(1-methylethyl)oxy]benzonitrile (Preparation 11) (176 mg, 0.544 mmol), tetrakis(triphenylphosphine)palladium(0) (62.8 mg, 0.054 mmol) and tripotassium phosphate (289 mg, 1.360 mmol) in DMF (4.5 ml) and water (0.900 ml) was stirred at 120° C. for 20 min under microwave irradiation then partitioned between water (50 ml) and AcOEt (50 ml). The two layers were separated and the aqueous phase extracted twice with AcOEt. The combined organic phases were washed with brine (2×50 ml), dried over MgSO₄ and concentrated in vacuo. Purification of the residue by flash chromatography on silica gel (c-Hexane/AcOEt: 0 to 50% gradient) gave 1,1-dimethylethyl 7-(5-{3-cyano-4-[(1-methylethyl)oxy]phenyl}-1,3,4-thiadiazol-2-yl)-1,2,4,5-tetrahydro-3H-3-benzazepine-3-carboxylate (246 mg, 88%) as a colourless solid.

LCMS: retention time 1.45 min; [M+H]⁺=491.19

### Preparation 16

### Ethyl 4-[7-(5-{3-cyano-4-[(1-methylethyl)oxy]phenyl}-1,3,4-thiadiazol-2-yl)-1,2,4,5-tetrahydro-3H-3-benzazepin-3-yl]butanoate

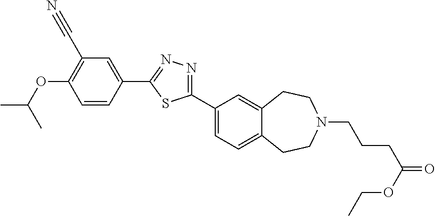

A slurry of 2-[(1-methylethyl)oxy]-5-[5-(2,3,4,5-tetrahydro-1H-3-benzazepin-7-yl)-1,3,4-thiadiazol-2-yl]benzonitrile hydrochloride (Example 6) (158 mg, 0.370 mmol) in DMF (3 ml) was treated with K₂CO₃ (205 mg, 1.480 mmol) then ethyl 4-bromobutyrate (0.064 ml, 0.444 mmol) and the resulting mixture was stirred at 100° C. for 9 hours then cooled to room temperature and concentrated in vacuo. The residue was partitioned between AcOEt and water and the two layers were separated. The organic phase was washed with water then brine, dried over MgSO₄ and concentrated in vacuo. Purification of the residue by flash chromatography on silica gel (c-Hexane/AcOEt: 0 to 100% gradient) gave ethyl 4-[7-(5-{3-cyano-4-[(1-methylethyl)oxy]phenyl}-1,3,4-thiadiazol-2-yl)-1,2,4,5-tetrahydro-3H-3-benzazepin-3-yl]butanoate (126 mg, 68%) as a yellow gum.

LCMS: retention time 0.98 min; [M+H]⁺=505.3

### Preparation 17

### 1,1-Dimethylethyl 5-methyl-6-oxo-3,4,6,7,8,8a-hexahydro-2(1H)-isoquinolinecarboxylate

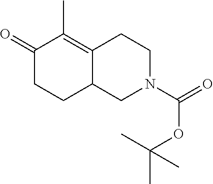

1,1-Dimethylethyl 4-oxo-1-piperidinecarboxylate (70 g, 351 mmol) and pyrrolidine (43.6 ml, 527 mmol) were dissolved in toluene (300 ml) and the resulting mixture was refluxed under a Dean and Stark apparatus for 24 hours then cooled to room temperature and concentrated in vacuo. The residue was dissolved in toluene (300 ml) and treated with hydroquinone (0.396 g) and 1-penten-3-one (29.6 g, 351 mmol). The resulting solution was refluxed for 24 hours then cooled to room temperature and dissolved with AcOEt (300 ml). The organic phase was washed with a 0.5N HCl aqueous solution (500 ml) and the aqueous phase extracted with AcOEt (300 ml). The combined organic phases were dried over MgSO₄ and concentrated in vacuo. Purification of the residue by flash chromatography on silica gel (c-Hexane/AcOEt: 0 to 60% gradient) gave 1,1-dimethylethyl 5-methyl-6-oxo-3,4,6,7,8,8a-hexahydro-2(1H)-isoquinolinecarboxylate (55.2 g, 59.2% yield) as pale yellow oil which crystallised on standing.

LCMS: retention time 1.05 min; [M+H]⁺=266.24

### Preparation 18

### 1,1-Dimethylethyl 6-hydroxy-5-methyl-3,4-dihydro-2(1H)-isoquinolinecarboxylate

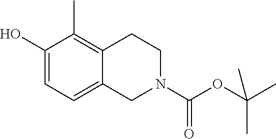

Lithium bis(trimethylsilyl)amide (1M in THF, 246 ml, 246 mmol) was added dropwise to a solution of 1,1-dimethylethyl 5-methyl-6-oxo-3,4,6,7,8,8a-hexahydro-2(1H)-isoquinolinecarboxylate (Preparation 17) (54.4 g, 205 mmol) in THF (200 ml) at −63° C., allowing a slight exotherm during the addition to −60° C. The mixture was stirred at this temperature for 30 minutes, then chloro(trimethyl)silane (31.4 ml, 246 mmol) was added dropwise. The resulting mixture was stirred for 2 hours at −70° C., then warmed to room temperature over 20 minutes and diluted with Et₂O (800 ml). The organic phase was added to a saturated Na₂CO₃ solution and the two layers were separated. The aqueous phase was extracted with Et₂O and the combined organic phases were dried over Na₂SO₄ and concentrated in vacuo. The residue was dissolved in CH₃CN (200 ml) and Palladium(II)acetate (46.0 g, 205 mmol) was added. The resulting mixture was cooled using a bowl of water to limit the exotherm to less than 35° C. and stirred for 16 hours. The insoluble material was filtered off through celite and rinsed with AcOEt (3×300 ml). The combined organic phases were then filtered through a 1 inch pad of silica gel and concentrated in vacuo. The residue was dissolved in AcOEt (500 ml) and treated with tetrabutylammonium fluoride (1M in THF, 200 ml, 200 mmol). The resulting mixture was allowed to stand for 30 minutes. The organic phase was then washed with a 0.5M HCl aqueous solution (300 ml) and a 10% w/w sodium thiosulphate aqueous solution, dried over MgSO₄ and concentrated in vacuo. Purification of the residue by flash chromatography on silica gel (c-Hexane/AcOEt: 0 to 60% gradient) gave 1,1-dimethylethyl 6-hydroxy-5-methyl-3,4-dihydro-2(1H)-isoquinolinecarboxylate (29.9 g, 55.4% yield) as a white solid.

LCMS: retention time 1.08 min; [M−H]⁻=262.37

### Preparation 19

### 1,1-Dimethylethyl 5-methyl-6-{[(trifluoromethyl)sulfonyl]oxy}-3,4-dihydro-2(1H)-isoquinolinecarboxylate

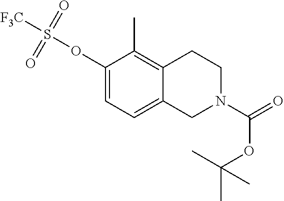

To a solution of 1,1-dimethylethyl 6-hydroxy-5-methyl-3,4-dihydro-2(1H)-isoquinolinecarboxylate (Preparation 18) (3.16 g, 12 mmol) in DCM (50 ml) at room temperature under nitrogen was added pyridine (1.94 ml, 24.00 mmol) and the resulting yellow solution was cooled to −30° C. before trifluoromethanesulfonic anhydride (2.23 ml, 13.20 mmol) was added dropwise. The resulting mixture was stirred 40 minutes at this temperature then warmed to room temperature and concentrated in vacuo. The residue was dissolved in AcOEt and the organic phase was washed sequentially with a 1N HCl aqueous solution, a saturated NaHCO₃ aqueous solution and brine, then dried over MgSO₄ and concentrated in vacuo to give 1,1-dimethylethyl 5-methyl-6-{[trifluoromethyl)sulfonyl]oxy}-3,4-dihydro-2(1H)-isoquinolinecarboxylate (4.85 g, 102%) as a red oil which was used in the next step without further purification.

LCMS: retention time 1.46 min; [M−H]⁻=394.22

### Preparation 20

### 1,1-Dimethylethyl 5-methyl-6-(4,4,5,5-tetramethyl-1,3,2-dioxaborolan-2-yl)-3,4-dihydro-2(1H)-isoquinolinecarboxylate

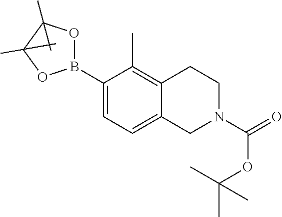

A solution of 1,1-dimethylethyl 5-methyl-6-{[trifluoromethyl)sulfonyl]oxy}-3,4-dihydro-2(1H)-isoquinolinecarboxylate (Preparation 19) (0.791 g, 2 mmol) in 1,4-dioxane (10 ml) was de-gassed under vacuum (ca 15 mbar) for 10 minutes then flushed with nitrogen. 1,1′-bis(diphenylphosphino)ferrocenedichloro palladium(II) (0.146 g, 0.200 mmol), potassium acetate (0.785 g, 8.00 mmol) then 4,4,4′,4′,5,5,5′,5′-octamethyl-2,2′-bi-1,3,2-dioxaborolane (0.609 g, 2.400 mmol) were added and the resulting red mixture was refluxed for 5 hours then cooled to room temperature and concentrated in vacuo. The residue was partitioned between AcOEt and water and the resulting biphasic mixture was filtered before the two layers were separated. The aqueous phase was extracted with AcOEt and the combined organic phases were washed with brine, dried over MgSO₄ and concentrated in vacuo. Purification of the residue by flash chromatography on silica gel (c-Hexane/AcOEt: 3 to 10% gradient) gave 1,1-dimethylethyl 5-methyl-6-(4,4,5,5-tetramethyl-1,3,2-dioxaborolan-2-yl)-3,4-dihydro-2(1H)-isoquinolinecarboxylate (660 mgs, 88%) as a colourless oil.

LCMS: retention time 1.56 min; [M+H]⁺=374.21

### Preparation 21

### 1,1-Dimethylethyl 6-(5-{3-cyano-4-[(1-methylethyl)oxy]phenyl}-1,3,4-thiadiazol-2-yl)-5-methyl-3,4-dihydro-2(1H)-isoquinolinecarboxylate

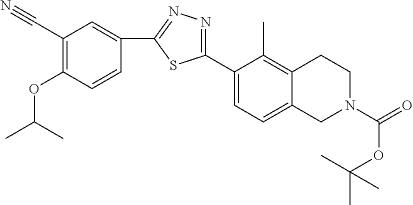

To a solution of 1,1-dimethylethyl 5-methyl-6-(4,4,5,5-tetramethyl-1,3,2-dioxaborolan-2-yl)-3,4-dihydro-2(1H)-isoquinolinecarboxylate (Preparation 20) in DME (3 ml) were added 5-(5-bromo-1,3,4-thiadiazol-2-yl)-2-[(1-methylethyl)oxy]benzonitrile (Preparation 11) (344 mg, 1.061 mmol), K₂CO₃ (611 mg, 4.42 mmol) then bis(triphenylphosphine)palladium(II) chloride (62.0 mg, 0.088 mmol) followed by water (1 ml) and the resulting mixture was stirred under nitrogen at 120° C. for 20 minutes under microwave irradiation. The two layers were separated and the aqueous phase was dissolved with water and extracted with AcOEt. The combined organic phases were washed with brine, dried over MgSO₄ and concentrated in vacuo. Purification of the residue by flash chromatography on silica gel (c-Hexane/AcOEt: 5 to 30% gradient) gave 1,1-dimethylethyl 6-(5-{3-cyano-4-[(1-methylethyl)oxy]phenyl}-1,3,4-thiadiazol-2-yl)-5-methyl-3,4-dihydro-2(1H)-isoquinolinecarboxylate (173 mg, 40%) as a white foam.

LCMS: retention time 1.45 min; [M+H]⁺=490.9

### Preparation 22

### Ethyl 3-[6-(5-{3-cyano-4-[(1-methylethyl)oxy]phenyl}-1,3,4-thiadiazol-2-yl)-5-methyl-3,4-dihydro-2(1H)-isoquinolinyl]propanoate

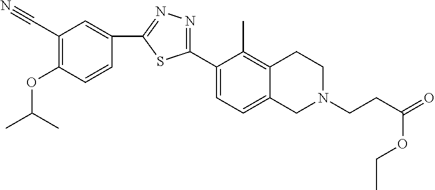

A flask was charged with 2-[(1-methylethyl)oxy]-5-[5-(5-methyl-1,2,3,4-tetrahydro-6-isoquinolinyl)-1,3,4-thiadiazol-2-yl]benzonitrile hydrochloride (Example 8) (854 mg, 2 mmol), ethyl acrylate (240 mg, 261 μl, 2.4 mmol) and 2,3,4,6,7,8,9,10-octahydropyrimido[1,2-a]azepine (914 mg, 904 μl, 6 mmol) then filled with CH₃CN (10 ml). The resulting suspension was stirred at room temperature for 4 hours then was diluted with AcOEt (20 ml). The organic phase was washed twice with water then brine, dried over MgSO₄ and concentrated in vacuo to give ethyl 3-[6-(5-{3-cyano-4-[(1-methylethyl)oxy]phenyl}-1,3,4-thiadiazol-2-yl)-5-methyl-3,4-dihydro-2(1H)-isoquinolinyl]propanoate (893 mgs, 91%) as a light brown solid which was used in the next step without further purification.

LCMS: retention time 0.96 min; [M+H]⁺=491.02

### Preparation 23

### Ethyl 4-[6-(5-{3-cyano-4-[(1-methylethyl)oxy]phenyl}-1,3,4-thiadiazol-2-yl)-5-methyl-3,4-dihydro-2(1H)-isoquinolinyl]butanoate

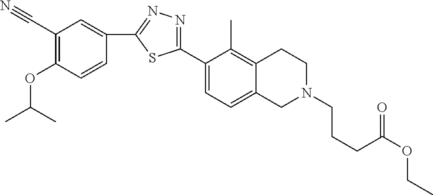

A mixture of 2-[(1-methylethyl)oxy]-5-[5-(5-methyl-1,2,3,4-tetrahydro-6-isoquinolinyl)-1,3,4-thiadiazol-2-yl]benzonitrile hydrochloride (Example 8) (854 mg, 2 mmol), K₂CO₃ (829 mg, 6 mmol) and ethyl 4-bromobutyrate (468 mg, 2.4 mmol) in dry DMF (5 ml) was stirred a 70° C. for 8 hours then cooled to room temperature and diluted with AcOEt (20 ml). The solution was washed with water (2×20 ml) then dried over MgSO₄ and concentrated in vacuo. Purification of the residue by flash chromatography on silica gel (DCM) gave ethyl 4-[6-(5-{3-cyano-4-[(1-methylethyl)oxy]phenyl}-1,3,4-thiadiazol-2-yl)-5-methyl-3,4-dihydro-2(1H)-isoquinolinyl]butanoate (650 mg, 64%) as a brown oil which solidified on standing.

LCMS: retention time 0.93 min; [M+H]⁺=505.0

### Preparation 24

### 1,1-Dimethylethyl 6-bromo-3,4-dihydro-2(1H)-isoquinolinecarboxylate

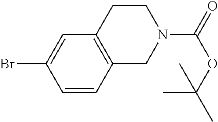

A suspension of 6-bromo-1,2,3,4-tetrahydroisoquinoline hydrochloride (CAS#226942-29-6, commercially available from Allichem LLC, 1 g, 4.02 mmol) and di-tert-butyl dicarbonate (1.87 ml, 8.05 mmol) in MeOH (10 ml) at room temperature under nitrogen was treated with triethylamine (2.80 ml, 20.12 mmol) and the resulting mixture was stirred at room temperature for 20 hours then concentrated in vacuo. The residue was partitioned between DCM and a saturated Na₂CO₃ aqueous solution and the two layers were separated using a phase separator cartridge. The organic phase was concentrated in vacuo and the residue loaded on a SCX column, eluting with MeOH. The combined methanolic fractions were concentrated in vacuo to give 1,1-dimethylethyl 6-bromo-3,4-dihydro-2(1H)-isoquinolinecarboxylate (1.215 g, 97%) as a pale yellow gum.

LCMS: retention time 1.38 min; [M+H]⁺=314.02 (1 Br)

### Preparation 25

### 1,1-Dimethylethyl 6-(4,4,5,5-tetramethyl-1,3,2-dioxaborolan-2-yl)-3,4-dihydro-2(1H)-isoquinolinecarboxylate

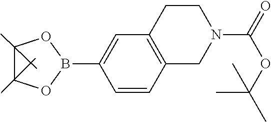

1,1-Dimethylethyl 6-bromo-3,4-dihydro-2(1H)-isoquinolinecarboxylate (Preparation 24) (1.215 g, 3.89 mmol) and 4,4,4′,4′,5,5,5′,5′-octamethyl-2,2′-bi-1,3,2-dioxaborolane (1.087 g, 4.28 mmol) were dissolved in 1,4-dioxane (15 ml) and the resulting mixture was de-gassed under vacuum (ca 15 mbar) then flushed with nitrogen. 1,1′-bis(diphenylphosphino)ferrocenedichloro palladium(II)dichloromethane complex (0.171 g, 0.234 mmol) and 1,1′-bis(diphenylphosphino)ferrocene (0.129 g, 0.234 mmol) were added. The resulting mixture was stirred at room temperature for 5 minutes, then treated with potassium acetate (1.146 g, 11.68 mmol), stirred at 90° C. under nitrogen for 4 hours then cooled to room temperature. The mixture was dissolved with a saturated Na₂CO₃ aqueous solution (20 ml) and the insoluble material was filtered off. The filtrate was extracted twice with AcOEt (20 ml) and the combined organic were washed with brine, dried over MgSO₄ and concentrated in vacuo. Purification of the residue by flash chromatography on silica gel (c-Hexane/AcOEt: 5 to 50% gradient) gave 1,1-dimethylethyl 6-(4,4,5,5-tetramethyl-1,3,2-dioxaborolan-2-yl)-3,4-dihydro-2(1H)-isoquinolinecarboxylate (580 mg, 41%) as a pale yellow oil which solidified on standing.

LCMS: retention time 1.42 min; no mass ion detected

### Preparation 26

### 1,1-Dimethylethyl 6-(5-{3-cyano-4-[(1-methylethyl)oxy]phenyl}-1,3,4-thiadiazol-2-yl)-3,4-dihydro-2(1H)-isoquinolinecarboxylate

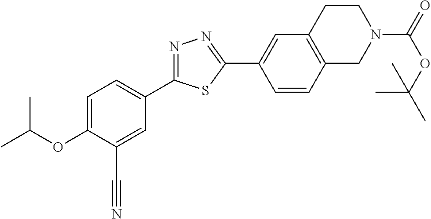

A suspension of 5-(5-bromo-1,3,4-thiadiazol-2-yl)-2-[(1-methylethyl)oxy]benzonitrile (Preparation 11) (497 mg, 1.534 mmol) and 1,1-dimethylethyl 6-(4,4,5,5-tetramethyl-1,3,2-dioxaborolan-2-yl)-3,4-dihydro-2(1H)-isoquinolinecarboxylate (Preparation 25) (580 mg, 1.614 mmol) in 1,4-dioxane (6 ml) and a saturated Na₂CO₃ aqueous solution (1.5 ml) was de-gassed under vacuum (ca 15 mbar) then flushed with nitrogen. 1,1′-bis(diphenylphosphino)ferrocenedichloro palladium(II)dichloromethane complex (118 mg, 0.161 mmol) was added. The resulting mixture was de-gassed under vacuum and flushed with nitrogen, stirred at 90° C. under nitrogen for 5 hours then cooled to room temperature and diluted with a saturated Na₂CO₃ aqueous solution and AcOEt. The insoluble material was filtered off and the layers were separated. The aqueous phase was extracted twice with AcOEt and the combined organic phases were washed with brine, dried over MgSO₄ and concentrated in vacuo. Purification of the residue by flash chromatography on silica gel (c-Hexane/AcOEt: 5 to 50% gradient) gave 1,1-dimethylethyl 6-(5-{3-cyano-4-[(1-methylethyl)oxy]phenyl}-1,3,4-thiadiazol-2-yl)-3,4-dihydro-2(1H)-isoquinolinecarboxylate (386 mg, 50%) as a yellow solid

LCMS: retention time 1.42 min; [M+H]⁺=477.0

### Preparation 27

### 1,1-Dimethylethyl 5-bromo-3,4-dihydro-2(1H)-isoquinolinecarboxylate

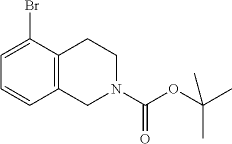

The title compound was obtained from 5-bromo-1,2,3,4-tetrahydroisoquinoline (CAS#81237-69-6, commercially available from Zannan Pharma) in an analogous manner to the procedure described for the synthesis of 1,1-dimethylethyl 6-bromo-3,4-dihydro-2(1H)-isoquinolinecarboxylate (Preparation 24).

LCMS: retention time 1.47 min; [M−H]⁻=310.98

### Preparation 28

### 1,1-Dimethylethyl 5-(4,4,5,5-tetramethyl-1,3,2-dioxaborolan-2-yl)-3,4-dihydro-2(1H)-isoquinolinecarboxylate

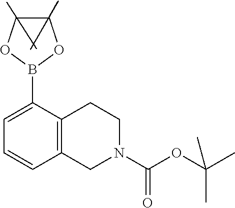

1,1-Dimethylethyl 5-bromo-3,4-dihydro-2(1H)-isoquinolinecarboxylate (Preparation 27) (0.281 g, 0.899 mmol), potassium acetate (0.265 g, 2.70 mmol), [1,1′-bis(diphenylphosphino)ferrocene]dichloropalladium(II) (0.066 g, 0.090 mmol) and 4,4,4′,4′,5,5,5′,5′-octamethyl-2,2′-bi-1,3,2-dioxaborolane (0.274 g, 1.079 mmol) were dissolved in 1,4-dioxane (5 ml) and the resulting mixture was stirred at 80° C. under nitrogen for 2 hours then cooled to room temperature and diluted with water (15 ml). The aqueous phase was extracted with AcOEt (3×10 ml). The combined organic phases were dried over MgSO₄ and concentrated in vacuo. Purification of the residue by flash chromatography on silica gel (c-Hexane/AcOEt: 15%) gave 1,1-dimethylethyl(4,4,5,5-tetramethyl-1,3,2-dioxaborolan-2-yl)-3,4-dihydro-2(1H)-isoquinolinecarboxylate 158 mg, 57%) as a light yellow oil.

LCMS: retention time 1.54 min; no mass ion detected.

### Preparation 29

### 1,1-Dimethylethyl 5-(5-{3-cyano-4-[(1-methylethyl)oxy]phenyl}-1,3,4-thiadiazol-2-yl)-3,4-dihydro-2(1H)-isoquinolinecarboxylate

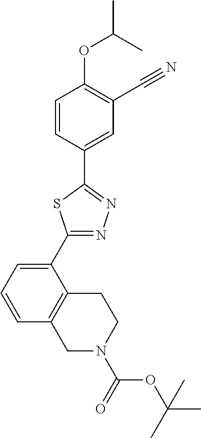

1,1-Dimethylethyl 5-(4,4,5,5-tetramethyl-1,3,2-dioxaborolan-2-yl)-3,4-dihydro-2(1H)-isoquinolinecarboxylate (Preparation 28) (153 mg, 0.426 mmol), 5-(5-bromo-1,3,4-thiadiazol-2-yl)-2-[(1-methylethyl)oxy]benzonitrile (Preparation 11) (115 mg, 0.355 mmol), dichlorobis(triphenylphosphine)-palladium (II) (24.90 mg, 0.035 mmol) and Na₂CO₃ (188 mg, 1.774 mmol) were dissolved in a mixture of DME (3.75 ml) and water (1.25 ml). The mixture was stirred at 120° C. for 20 minutes under microwave irradiation. The mixture was diluted with water (20 ml) and the aqueous phase was extracted with AcOEt (2×15 ml). The combined organic phases were dried under Na₂SO₄ and concentrated in vacuo. Purification of the residue by flash chromatography on silica gel (c-Hexane/AcOEt: 0 to 20% gradient) gave 1,1-dimethylethyl 5-(5-{3-cyano-4-[(1-methylethyl)oxy]phenyl}-1,3,4-thiadiazol-2-yl)-3,4-dihydro-2(1H)-isoquinolinecarboxylate (105 mg, 62%) as a light yellow oil.

LCMS: retention time 1.47 min; [M+H]⁺=477.01

### Preparation 30

### 1,3-dibromo-2-ethylbenzene

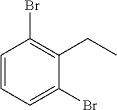

A three necked round-bottom flask was purged with argon and then filled with dry THF (60 mL), 1,3-dibromo benzene (9.5 g, 40.27 mmol) and ethyl iodide (8.8 g, 56.38 mmol). The mixture was cooled to −78° C. and LDA [made from 8 mL of iPr₂NH and 10 mL of BuLi (2.5M in hexane) in 40 mL of THF] was added slowly at −70° C. After stirring for 2 hr the reaction was poured into 100 mL of sat. aq. NH₄Cl solution and stirred vigorously for 20 min, extracted with DCM (2*100 mL). The organic layer was evaporated to give the crude product, which was purified by column chromatography with hexane to give 1,3-Dibromo-2-ethylbenzene (8.5 g, 79.9%). δH(CDCl₃, 400 MHz): 7.76-7.78 (2H, m), 6.86-6.90 (1H, m), 2.89-3.01 (2H, q), 1.12 (3H, t) ppm. MS (ES⁺): C₈H₈Br₂ requires 264; found 265 (M+H⁺).

### Preparation 40

### 3-bromo-2-ethyl-benzaldehyde

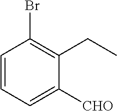

To a solution of 1,3-dibromo-2-ethyl-benzene (24 g, 90.92 mmol) in THF (300 mL) was added 36.4 mL of BuLi (2.5 M in hexane, 90.92 mmol) under N₂. The mixture was stirred for 2 hr at −78° C. Then DMF (12 g, 163.66 mmol) was added, after stirring for 2 hr, the reaction was poured onto 300 mL of sat. aq. NH₄Cl solution and extracted with DCM (2*100 mL). The organic layer was evaporated to give the crude product, which was purified by column chromatography eluting with EtOAc:hexane (1:20) to give 3-Bromo-2-ethylbenzaldehyde (7.6 g, yield 39.22%). δH(CDCl₃, 400 MHz): 10.25 (1H, s), 7.76-7.78 (2H, m), 7.10-7.22 (1H, m), 3.20-3.27 (2H, q), 1.20 (3H, q) ppm. MS (ES⁺): C₉H₉BrO requires 213; found 214 (M+H⁺).

### Preparation 41

### 1-bromo-2-ethyl-3-(2-nitrovinyl)benzene

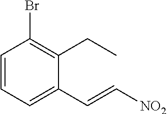

A mixture of 3-bromo-2-ethylbenzaldehyde (65.0 g, 307 mmol) and NH₄OAc (12.0 g, 154 mmol) in 200 mL of CH₃NO₂ was refluxed for 3 hours. The solvent was concentrated and purified on silica gel to give the desired product (66.9 g, 88.5% yield).

δH(CDCl₃, 400 MHz): 8.20 (1H, d), 7.59 (1H, d), 7.36-7.40 (1H, m), 7.02-7.06 (1H, m), 2.87-2.93 (4H, q), 1.12 (3H, t) ppm. MS (ES⁺): C₁₀H₁₀BrNO₂ requires 256; found 257 (M+H⁺).

### Preparation 42

### 2-(3-bromo-2-ethylphenyl)ethylamine

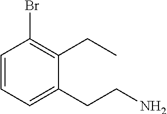

To a mixture of LiBH₄ (11.0 g, 500 mmol) and 300 mL of THF was added TMSCl (108 g, 1 mol) at 0° C. The reaction was stirred at 0° C. for 10 min, then added a solution of 1-bromo-2-ethyl-3-(2-nitrovinyl)benzene (25.5 g, 100 mmol) in 100 mL of THF. The reaction mixture was warmed to room temperature and refluxed for 3 hours. After cooling to room temperature, 40 mL of MeOH was added carefully. The solvent was concentrated, and diluted with 300 mL of EtOAc and 100 mL of water. The organic layer was dried over Na₂SO₄ and concentrated to give the crude product (19.0 g, 84.0% yield).

δH(CDCl₃, 400 MHz): 8.30 (2H, brs), 7.33-7.36 (1H, m), 7.04-7.19 (1H, m), 6.85-6.89 (1H, m), 3.09-3.19 (4H, m), 2.72-2.78 (2H, m), 1.08 (3H, t) ppm. MS (ES⁺):

C₁₀H₁₄BrN requires 227; found 228 (M+H⁺).

### Preparation 43

### N-[2-(3-bromo-2-ethylphenyl)ethyl]-2,2,2-trifluoroacetamide

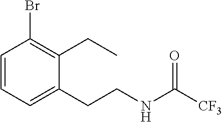

A mixture of 2-(3-bromo-2-ethylphenyl)ethylamine (19.0 g, 83.7 mmol), Et₃N (16.9 g, 167.4 mmol) and dry DCM (200 mL) was cooled to 0° C. Trifluoroacetic anhydride (35.2 g, 167.4 mmol) was added dropwise. The reaction was warmed to room temperature and allowed to stir for 16 hours. Water (100 mL) was added. The reaction was extracted with DCM (3×150 mL). The organic layer was dried over Na₂SO₄ and concentrated. Purification was performed on silica gel to give the desired product (25.79, 95.4% yield).

δH(CDCl₃, 400 MHz): 7.45 (2H, d), 6.90-7.10 (2H, m), 3.51-3.60 (2H, m), 2.64-2.95 (4H, m), 1.22 (3H, t) ppm. MS (ES⁺): C₁₂H₁₃BrF₃NO requires 323; found 324 (M+H⁺).

### Preparation 44

### 1-(6-bromo-5-ethyl-3,4-dihydro-1H-isoquinolin-2-yl)-2,2,2-trifluoroethanone

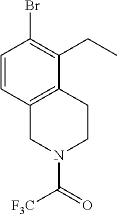

A mixture of glacial acetic (100 mL), concentrated sulfuric acid (40 mL), N-[2-(3-bromo-2-ethyl-phenyl)ethyl]-2,2,2-trifluoro-acetamide (25.0 g, 77.4 mmol) and (CH₂O)n (2.5 g) was heated at 70° C. for 4 hours, and then cooled to room temperature. The reaction mixture was poured into 200 mL of cold water, and extracted with EtOAc (3*200 mL). The organic layer was dried over Na₂SO₄ and concentrated. Purification was performed on silica gel to give the desired product. δH(CDCl₃, 400 MHz): 7.41 (2H, d), 6.81 (2H, d), 4.66 (2H, d), 3.83-3.86 (2H, m), 2.94-2.98 (2H, m), 2.77-2.88 (2H, m), 1.19 (3H, t) ppm. MS (ES⁺): C₁₃H₁₃BrF₃NO requires 335; found 336 (M+H⁺).

### Preparation 45

### 1-[5-ethyl-6-(4,4,5,5-tetramethyl-[1,3,2]dioxaborolan-2-yl)-3,4-dihydro-1H-isoquinolin-2-yl]-2,2,2-trifluoroethanone

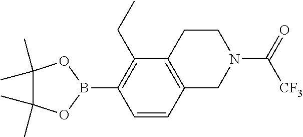

A mixture of 1-(6-bromo-5-ethyl-3,4-dihydro-1H-isoquinolin-2-yl)-2,2,2-trifluoroethanone (6.0 g, 17.9 mmol), 4,4,5,5,4′,4′,5′,5′-octamethyl-[2,2′]bi[[1,3,2]dioxaborolanyl] (5.46 g, 21.5 mmol), Pd(dppf)Cl₂ (1.31 g, 1.79 mmol) and CH₃COOK (3.5 g, 35.8 mmol) and 150 mL of 1,4-dioxane was refluxed for 10 hours. The reaction mixture was concentrated and purified on silica gel to give the desired product.

δH (CDCl₃, 400 MHz): 7.57 (2H, d), 6.91 (2H, d), 4.66 (2H, d), 3.79-3.82 (2H, m), 2.91-2.95 (4H, m), 1.25 (12H, s), 1.09 (3H, t) ppm. MS (ES⁺): C₁₉H₂₅BF₃NO₃ requires 383; found 384 (M+H⁺).

### Preparation 46

### 2-hydroxy-5-iodobenzonitrile

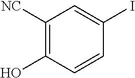

To a solution of 2-hydroxybenzonitrile (20 g, 0.168 mol) in CH₃CN (200 mL) was added dropwise CF₃SO₃H (16.5 mL) at 0° C. and followed by addition of NIS (45.4 g, 0.201 mol) at 0° C. The solution was stirred at rt overnight. The reaction mixtue was concentrated, washed with H₂O (1 L), extracted with DCM (800 mL*3). The combined organic layer was dried and concentrated. Purification was performed by column chromatography (PE:EA=10:1) to get 30 g of 2-hydroxy-5-iodo-benzonitrile (yield 73.2%).

δH (DMSO, 400 MHz): 7.68 (1H, s), 7.60-7.58 (1H, d), 6.67-6.65 (1H, d) ppm. MS (ES⁺): C₇H₄INO requires 245; found 246 (M+H⁺).

### Preparation 46

### 5-iodo-2-isopropoxybenzonitrile

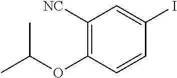

A mixture of 2 (42 g, 0.173 mol), iPrl (58.7 g, 0.345 mol) and K₂CO₃ (47.7 g 0.345 mol) and CH₃CN (420 mL) was stirred at reflux overnight. The reaction mixture was filtered and concentrated. The residue was diluted with DCM (300 mL) and filtered. The filtrate was concentrated to get 48.5 g of 5-iodo-2-isopropoxybenzonitrile (yield 97.6%)

δH(CDCl₃, 400 MHz): 7.79 (1H, s), 7.75-7.73 (1H, d), 6.74-6.72 (1H, d), 4.62-4.59 (1H, m), 1.39-1.37 (6H, s) ppm. MS (ES⁺): C₁₀H₁₀INO requires 287; found 288 (M+H⁺).

### Preparation 47

### 2-isopropoxy-5-(4,4,5,5-tetramethyl-[1,3,2]dioxaborolan-2-yl)benzonitrile

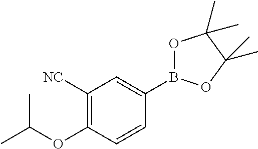

A mixture of 5-iodo-2-isopropoxybenzonitrile (15 g, 0.052 mol), Pin₂B₂ (15.9 g, 0.0627 mol), Pd(dppf)Cl₂ (4.3 g, 5.2 mmol) and KOAc (15.3 g, 0.156 mol) and dioxane (150 mL) was stirred at 90° C. under N₂ overnight. The reaction mixture was filtered and concentrated. Purification was performed by column chromatography (PE:EA=200:1) to get 13.8 g of 2-isopropoxy-5-(4,4,5,5-tetramethyl-[1,3,2]dioxaborolan-2-yl)-benzonitrile (yield 92%).

δH(CDCl₃, 400 MHz): 7.9-8.0 (1H, d), 7.85-7.89 (1H, dd), 6.92-6.90 (1H, d), 4.65-4.74 (1H, m), 1.39-1.37 (6H, s), 1.24 (6H, s) ppm. MS (ES⁺): C₁₆H₂₂BNO₃ requires 287; found 288 (M+H⁺).

### Preparation 48

### 2-isopropoxy-5-thiazol-2-ylbenzonitrile

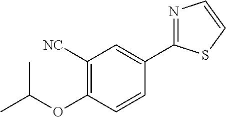

A mixture of 2-isopropoxy-5-(4,4,5,5-tetramethyl-[1,3,2]dioxaborolan-2-yl)benzonitrile (5 g, 17.4 mmol), 2-bromo-thiazole (3.43 g, 20.9 mmol), Pd(dppf)Cl₂ (1.4 g, 1.74 mmol) and Na₂CO₃(3.7 g, 34.8 mmol) and DME/H₂O (100 mL, 1:1) was stirred at reflux under N₂ overnight. The reaction mixture was cooled to rt, extracted with DCM (300 mL*3). The organic layer was dried and concentrated. Purification was performed by column chromatography (PE:EA=10:1) to get 2 g of 2-isopropoxy-5-thiazol-2-yl-benzonitrile (yield 47.1%).

δH(CDCl₃, 400 MHz): 8.13 (1H, s), 8.10-8.17 (1H, d), 7.83-7.82 (1H, d), 7.32-7.31 (1H, d), 7.03-7.00 (1H, d), 4.73-4.70 (1H, m), 1.43-1.41 (6H, s) ppm. MS (ES⁺): C₁₃H₁₂N₂OS requires 244; found 245 (M+H⁺).

### Preparation 49

### 5-(5-bromo-thiazol-2-yl)-2-isopropoxybenzonitrile

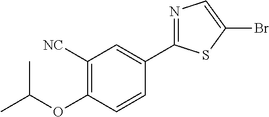

To a mixture of 2-isopropoxy-5-thiazol-2-ylbenzonitrile (1.88 g, 7.7 mmol) and DMF (19 mL) was added NBS (2.74 g, 15.4 mmol) at 0° C. After addition the resulting mixture was stirred at it for 3 h. H₂O (100 mL) was added. The reaction mixture was extracted with DCM (100 mL*3), washed by water (100 mL*3). The organic phase was separated, dried and concentrated. Purification was performed by column chromatography (PE:EA=20:1) to get 1.9 g of 5-(5-bromo-thiazol-2-yl)-2-isopropoxy-benzonitrile (yield 76.3%). MS (ES⁺): C₁₃H₁₁BrN₂OS requires 322; found 323 (M+H⁺).

### Preparation 50

### 5-[5-(5-ethyl-3,4-dihydro-isoquinolin-6-yl)-thiazol-2-yl]-2-isopropoxybenzonitrile

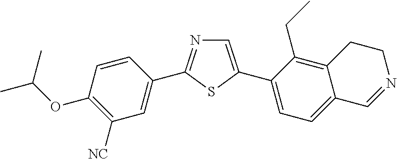

A mixture of 5-(5-bromo-thiazol-2-yl)-2-isopropoxybenzonitrile (400 mg, 1.24 mmol), 1-[5-ethyl-6-(4,4,5,5-tetramethyl-[1,3,2]dioxaborolan-2-yl)-3,4-dihydro-1H-isoquinolin-2-yl]-2,2,2-trifluoroethanone (570 mg, 1.49 mmol), Pd(dppf)Cl₂ (101 mg, 0.124 mmol) and Na₂CO₃ (262 mg, 2.48 mmol) and DME/H₂O (8 mL, 1:1) was stirred at reflux under N₂ overnight. The reaction mixture was cooled to rt, extracted with DCM (20 mL*3). The combined organic layer was dried and concentrated. Purification was performed by prep-HPLC to get 260 mg of 5-[5-(5-ethyl-3,4-dihydro-isoquinolin-6-yl)-thiazol-2-yl]-2-isopropoxybenzonitrile (yield 52.2%).

δH(CDCl₃, 400 MHz): 8.15-8.10 (2H, m), 7.82-7.70 (1H, d), 7.55-7.54 (1H, m), 7.11-7.08 (2H, d), 7.28-7.26 (1H, d), 7.08-7.01 (2H, m), 4.76-4.72 (1H, m), 4.40 (1H, s), 4.10-4.08 (1H, d), 3.54 (1H, 1), 3.25-3.23 (2H, d), 2.85-2.84 (1H, m), 2.71-2.69 (1H, m), 1.46-1.43 (6H, m), 1.21-1.10 (3H, m) ppm. MS (ES⁺): C₂₄H₂₃N₃OS requires 401; found 402 (M+H⁺).

### Preparation 51

### 5-[5-(5-ethyl-1,2,3,4-tetrahydro-isoquinolin-6-yl)-thiazol-2-yl]-2-isopropoxybenzonitrile

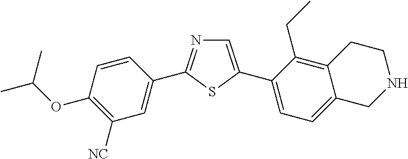

A solution of 5-[5-(5-ethyl-3,4-dihydro-isoquinolin-6-yl)-thiazol-2-yl]-2-isopropoxy-benzo nitrile (250 mg, 0.62 mmol) and NaBH₄ (70 mg, 1.24 mmol) in EtOH (5 mL) was stirred at reflux for 2 h. The reaction mixture was concentrated, diluted with DCM (30 mL), filtered, dried over Na₂SO₄ and concentrated. Purification was performed by prep-HPLC to get 100 mg of 5-[5-(5-Ethyl-1,2,3,4-tetrahydro-isoquinolin-6-yl)-thiazol-2-yl]-2-isopropoxy-benzo nitrile (yield 40%).

δH(CDCl₃, 400 MHz): 8.12 (1H, s), 8.10-8.09 (1H, s), 7.70 (1H, s), 7.29-7.25 (1H, d), 7.28-7.26 (1H, d), 7.08-7.02 (2H, m), 4.77-4.71 (1H, m), 4.39 (2H, m), 3.60-3.48 (2H, m), 3.40-3.06 (6H, m), 2.75-2.68 (2H, m), 1.51-1.44 (6H, s), 1.14-1.10 (3H, m) ppm. MS (ES⁺): C₂₄H₂₅N₃OS requires 403; found 404 (M+H⁺).

### Preparation 52

### 5-{3-chloro-4-[(1-methylethyl)oxy]phenyl}-1,3,4-thiadiazol-2-amine

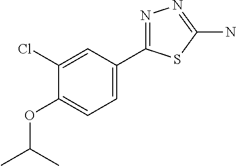

The mixture of 3-chloro-4-[(1-methylethyl)oxy]benzoic acid (5 g), hydrazinecarbothioamide (2.229 g) in phosphoric trichloride (21.43 g) was stirred at 75° C. for 3 h. Concentrated the mixture in vacuum to remove POCl₃, the residue was poured into crush ice. Basified the mixture with aqueous NaOH. Extracted it with EA twice. The EA layer was concentrated in vacuum to give crude product 5-{3-chloro-4-[(1-methylethyl)oxy]phenyl}-1,3,4-thiadiazol-2-amine (7.5 g), which was used in the next step without further purification. MS (ES): C₁₁H₁₂ClN₃OS requires 269; found 270.1 (M+H⁺).

### Preparation 53

### 2-bromo-5-{3-chloro-4-[(1-methylethyl)oxy]phenyl}-1,3,4-thiadiazole

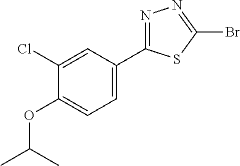

The mixture of 5-{3-chloro-4-[(1-methylethyl)oxy]phenyl}-1,3,4-thiadiazol-2-amine (6.25 g), copper(II) bromide (10.35 g) in acetonitrile (60 mL) was stirred at room temperature, then 1,1-dimethylethyl nitrite (5.51 mL) was added. The mixture was stirred at room temperature for 3 h. It was treated with EA and aqueous HCl, the EA layer was combined and purified by column chromatography to give 2-bromo-5-{3-chloro-4-[(1-methylethyl)oxy]phenyl}-1,3,4-thiadiazole (4.76 g). MS (ES): C₁₁H₁₀BrClN₂OS requires 333; found 334.0 (M+H⁺).

### Preparation 54

### 5-[5-(5-ethyl-1,2,3,4-tetrahydro-isoquinolin-6-yl)-thiazol-2-yl]-2-isopropoxybenzonitrile

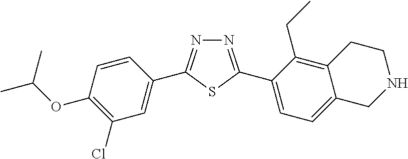

A mixture of 2-bromo-5-{3-chloro-4-[(1-methylethyl)oxy]phenyl}-1,3,4-thiadiazole (400 mg, 1.20 mmol), 1-[5-ethyl-6-(4,4,5,5-tetramethyl-[1,3,2]dioxaborolan-2-yl)-3,4-dihydro-1H-isoquinolin-2-yl]-2,2,2-trifluoroethanone (570 mg, 1.49 mmol), Pd(dppf)Cl₂ (101 mg, 0.124 mmol) and Na₂CO₃ (262 mg, 2.48 mmol) and DME/H₂O (8 mL, 1:1) was stirred at reflux under N₂ overnight. The reaction mixture was cooled to rt, extracted with DCM (20 mL*3). The combined organic layer was dried and concentrated. Purification was performed by prep-HPLC to get 260 mg of 5-[5-(5-ethyl-1,2,3,4-tetrahydro-isoquinolin-6-yl)-thiazol-2-yl]-2-isopropoxybenzonitrile (yield 52%).

MS (ES⁺): C₂₂H₂₃ClN₃OS requires 413; found 414 (M+H⁺).

### Preparation 55

### Methyl 3-cyano-4-{[(trifluoromethyl)sulfonyl]oxy}benzoate

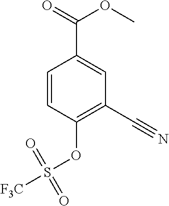

To a solution of methyl 3-cyano-4-hydroxybenzoate (3 g, 16.93 mmol) and triethylamine (3.54 ml, 25.4 mmol) in dry dichloromethane (60 ml) at 0° C. under a flush of argon was added trifluoromethanesulfonic anhydride (3.15 ml, 18.63 mmol) slowly dropwise. The reaction was allowed to warm to room temperature and stirred for 1 h. The reaction mixture was washed with 10% aqueous potassium carbonate (2×50 mL) and then aqueous HCl (2M, 2×50 mL) before the organic phase was dried (phase separator) and the solvent removed in vacuo to give the title compound as a dark brown oil, (5.165 g, 16.70 mmol). δH(CDCl₃, 400 MHz): 8.44 (1H, d), 8.38 (1H, dd), 7.60 (1H, d), 3.99 (3H, s). MS (ES): no mass ion observed.

### Preparation 56

### Methyl 3-cyano-4-(2-methylpropyl)benzoate

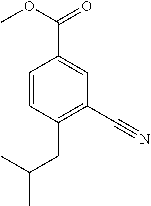

To methyl 3-cyano-4-{[(trifluoromethyl)sulfonyl]oxy}benzoate (1.5 g, 4.85 mmol) was added bromo(2-methylpropyl)zinc (48.5 ml, 24.25 mmol) in tetrahydrofuran (50 ml) under argon. To the solution was then added 1,1′-bis(diphenylphosphino)ferrocenedichloro palladium(II)dichloromethane complex (0.355 g, 0.485 mmol) and the reaction heated to reflux for 6 h. The mixture was quenched with water (2 mL) and then filtered through celite, washing with ethyl acetate. The solvent was removed in vacuo. The residue was partitioned between ethyl acetate (50 mL) and water (50 mL) and the organic phase dried (phase separator) and the solvent removed in vacuo. The residue was purified by silica chromatography, eluting 0-15% EtOAc in iso-hexane over 40 min. Two batches were collected, one of which was the title compound as a colourless oil (233 mg, 1.072 mmol). δH(CDCl₃, 400 MHz): 8.28 (1H, d), 8.15 (1H, dd), 7.38 (1H, d), 3.94, 3H, s), 2.78 (2H, d), 2.02 (1H, m), 0.96 (6H, d).

### Preparation 57

### 3-Cyano-4-(2-methylpropyl)benzoic acid

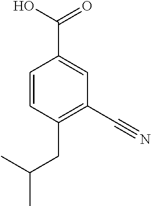

Methyl 3-cyano-4-(2-methylpropyl)benzoate (233 mg, 1.072 mmol) was dissolved in ethanol (4 ml) and 2M aqueous sodium hydroxide (1 ml, 2 mmol) was added. The reaction was stirred for 1 h. 2M aqueous HCl (10 mL) was added and the mixture extracted with dichloromethane (20 mL+10 mL). The organic phases were isolated and dried by phase separator and combined before the solvent was removed in vacuo to give the title compound as a white solid (203 mg, 0.999 mmol). δH (d₆-DMSO, 400 MHz) 13.43 (1H, br. s), 8.29 (1H, d), 8.14 (1H, dd), 7.59 (1H, d), 2.74 (2H, d), 1.96 (1H, m), 0.91 (6H, d). MS (ES): C₁₂H₁₃NO₂ requires 203; found 202 (M−H⁺).

### Preparation 58

### 5-(5-amino-1,3,4-thiadiazol-2-yl)-2-(2-methylpropyl)benzonitrile

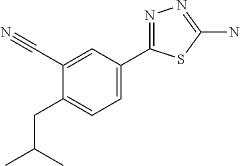

The mixture of 3-cyano-4-(2-methylpropyl)benzoic acid (5 g), hydrazinecarbothioamide (2.229 g) in phosphoric trichloride (21.43 g) was stirred at 75° C. for 3 h. Concentrated the mixture in vacuum to remove POCl₃, the residue was poured into crush ice. Basified the mixture with aqueous NaOH. Extracted it with EA twice. The EA layer was concentrated in vacuum to give crude product 5-(5-amino-1,3,4-thiadiazol-2-yl)-2-(2-methylpropyl)benzonitrile (7.5 g), which was used in the next step without further purification. MS (ES): C₁₃H₁₄N₄S requires 258; found 259.1 (M+H⁺).

### Preparation 59

### 5-(5-bromo-1,3,4-thiadiazol-2-yl)-2-(2-methylpropyl)benzonitrile

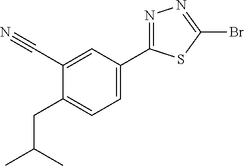

The mixture of 5-(5-amino-1,3,4-thiadiazol-2-yl)-2-(2-methylpropyl)benzonitrile (6.25 g), copper(II) bromide (10.35 g) in acetonitrile (60 mL) was stirred at room temperature, then 1,1-dimethylethyl nitrite (5.51 mL) was added. The mixture was stirred at room temperature for 3 h. It was treated with EA and aqueous HCl, the EA layer was combined and purified by column chromatography to give 5-(5-bromo-1,3,4-thiadiazol-2-yl)-2-(2-methylpropyl)benzonitrile (4.76 g). MS (ES): C₁₃H₁₂BrN₃S requires 322; found 323.0 (M+H⁺).

### Preparation 60

### 5-[5-(5-ethyl-1,2,3,4-tetrahydro-6-isoquinolinyl)-1,3,4-thiadiazol-2-yl]-2-(2-methylpropyl)benzonitrile

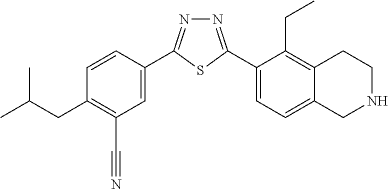

A mixture of 5-(5-bromo-1,3,4-thiadiazol-2-yl)-2-(2-methylpropyl)benzonitrile (400 mg, 1.20 mmol), 1-[5-ethyl-6-(4,4,5,5-tetramethyl-[1,3,2]dioxaborolan-2-yl)-3,4-dihydro-1H-isoquinolin-2-yl]-2,2,2-trifluoroethanone (570 mg, 1.49 mmol), Pd(dppf)Cl₂ (101 mg, 0.124 mmol) and Na₂CO₃ (262 mg, 2.48 mmol) and DME/H₂O (8 mL, 1:1) was stirred at reflux under N₂ overnight. The reaction mixture was cooled to rt, extracted with DCM (20 mL*3). The combined organic layer was dried and concentrated. Purification was performed by prep-HPLC to get 260 mg of 5-[5-(5-ethyl-1,2,3,4-tetrahydro-6-isoquinolinyl)-1,3,4-thiadiazol-2-yl]-2-(2-methylpropyl)benzonitrile (yield 52%).

MS (ES⁺): C₂₄H₂₆N₄S requires 402; found 403 (M+H⁺).

### EXAMPLE 1

### 7-(2-{3-Chloro-4-[(1-methylethyl)oxy]phenyl}-1,3-thiazol-5-yl)-2,3,4,5-tetrahydro-1H-3-benzazepine hydrochloride

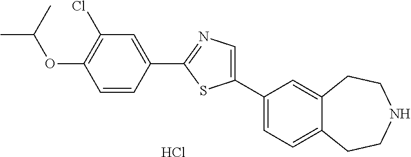

1,1-Dimethylethyl 7-(2-{3-chloro-4-[(1-methylethyl)oxy]phenyl}-1,3-thiazol-5-yl)-1,2,4,5-tetrahydro-3H-3-benzazepine-3-carboxylate (Preparation 4) (463 mg, 0.826 mmol) in 1,4-dioxane (4 ml) at room temperature was treated with a 4M HCl solution in 1,4-dioxane (4 ml, 16.00 mmol). After 2 hours, the reaction mixture was diluted with Et₂O (10 ml). The precipitate formed was filtered off and rinsed with Et₂O then sonicated as a suspension in acetone. The precipitate was filtered off and rinsed with acetone to give 7-(2-{3-chloro-4-[(1-methylethyl)oxy]phenyl}-1,3-thiazol-5-yl)-2,3,4,5-tetrahydro-1H-3-benzazepine hydrochloride (62 mg, 17%) as a lemon yellow solid.

The combined organic layers were concentrated in vacuo and the residue was loaded onto a SCX cartridge, eluting first with MeOH, then with a 2M NH₃ solution in MeOH. The ammonia fractions were combined and concentrated in vacuo to give 7-(2-{3-chloro-4-[(1-methylethyl)oxy]phenyl}-1,3-thiazol-5-yl)-2,3,4,5-tetrahydro-1H-3-benzazepine (256 mg, 77%) as a pale yellow foam.

LCMS: Retention time 1.10 min, [M+H]⁺=399.3

### EXAMPLE 2

### 7-(5-{3-Chloro-4-[(1-methylethyl)oxy]phenyl}-1,3-thiazol-2-yl)-2,3,4,5-tetrahydro-1 H-3-benzazepine hydrochloride

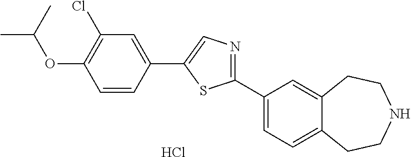

A solution of 1,1-dimethylethyl 7-(5-{3-chloro-4-[(1-methylethyl)oxy]phenyl}-1,3-thiazol-2-yl)-1,2,4,5-tetrahydro-3H-3-benzazepine-3-carboxylate (Preparation 7) (289 mg, 0.579 mmol) in DCM (4 ml) at room temperature was treated with trifluoroacetic acid (2 ml, 26.0 mmol) and the resulting mixture was stirred for 1 hour at this temperature then concentrated in vacuo. The residue dissolved in MeOH was loaded on a SCX cartridge pre-washed with MeOH and was eluted with MeOH then with a 2N NH₃ solution in MeOH. The ammonia fractions were combined and concentrated in vacuo to give 7-(5-{3-chloro-4-[(1-methylethyl)oxy]phenyl}-1,3-thiazol-2-yl)-2,3,4,5-tetrahydro-1H-3-benzazepine (226 mgs, 98%) as a pale yellow gum. 15 mgs of the amine were dissolved in MeOH (0.5 ml) and treated with a 1.35M HCl solution in MeOH (0.5 ml) and the resulting mixture was concentrated in vacuo. The residue was triturated with Et₂O to give 7-(5-{3-chloro-4-[(1-methylethyl)oxy]phenyl}-1,3-thiazol-2-yl)-2,3,4,5-tetrahydro-1H-3-benzazepine hydrochloride as a yellow solid.

LCMS: retention time 1.14 min; [M+H]⁺=399.17

### EXAMPLE 3

### 4-[7-(2-{3-Chloro-4-[(1-methylethyl)oxy]phenyl}-1,3-thiazol-5-yl)-1,2,4,5-tetrahydro-3H-3-benzazepin-3-yl]butanoic acid

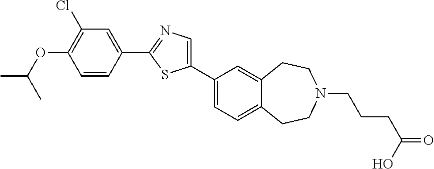

A suspension of ethyl 4-[7-(2-{3-chloro-4-[(1-methylethyl)oxy]phenyl}-1,3-thiazol-5-yl)-1,2,4,5-tetrahydro-3H-3-benzazepin-3-yl]butanoate (Preparation 8) (268 mg, 0.522 mmol) in EtOH (7 ml) was treated with a 2N NaOH aqueous solution (0.522 ml, 1.045 mmol) and the resulting mixture was stirred at room temperature for 16 hours then concentrated in vacuo. The residue was dissolved in water and the aqueous phase was acidified (pH 6) with a 2N HCl aqueous solution then extracted with AcOEt to which a few drops of MeOH were added in order to aid solubility. The organic phase was dried under MgSO₄ then concentrated in vacuo. Trituration of the residue with Et₂O gave 4-[7-(2-{3-chloro-4-[(1-methylethyl)oxy]phenyl}-1,3-thiazol-5-yl)-1,2,4,5-tetrahydro-3H-3-benzazepin-3-yl]butanoic acid (42 mgs, 16%) as a cream coloured solid.

LCMS: retention time 1.09 min; [M+H]⁺=485.3

### EXAMPLE 4

### 4-[7-(5-{3-Chloro-4-[(1-methylethyl)oxy]phenyl}-1,3-thiazol-2-yl)-1,2,4,5-tetrahydro-3H-3-benzazepin-3-yl]butanoic acid

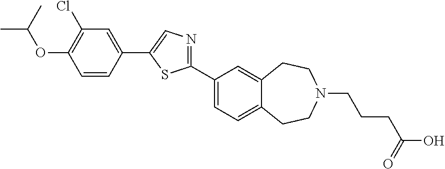

A solution of ethyl 4-[7-(5-{3-chloro-4-[(1-methylethyl)oxy]phenyl}-1,3-thiazol-2-yl)-1,2,4,5-tetrahydro-3H-3-benzazepin-3-yl]butanoate (Preparation 9) (209 mg, 0.407 mmol) in EtOH (3 ml), at room temperature was treated with a 2N NaOH aqueous solution (0.407 ml, 0.815 mmol). The resulting mixture was stirred at room temperature for 20 hours. Acetic acid (0.047 ml, 0.815 mmol) was then added and the resulting solution was concentrated in vacuo then co-evaporated with EtOH. The residue was triturated with Et₂O then dried at 60° C. under vacuum (ca 15 mbar) for 48 hours to give 4-[7-(5-{3-chloro-4-[(1-methylethyl)oxy]phenyl}-1,3-thiazol-2-yl)-1,2,4,5-tetrahydro-3H-3-benzazepin-3-yl]butanoic acid:AcOEt (1:2) (174 mgs, 66%) as a cream coloured amorphous solid.

LCMS: retention time 1.13 min; [M+H]⁺=485.17

### EXAMPLE 5

### 7-(5-{3-Chloro-4-[(1-methylethyl)oxy]phenyl}-1,3,4-thiadiazol-2-yl)-2,3,4,5-tetrahydro-1H-3-benzazepine hydrochloride

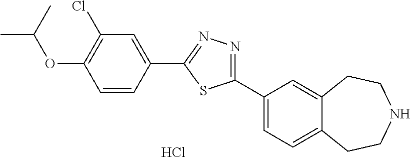

1,1-Dimethylethyl 7-(5-{3-chloro-4-[(1-methylethyl)oxy]phenyl}-1,3,4-thiadiazol-2-yl)-1,2,4,5-tetrahydro-3H-3-benzazepine-3-carboxylate (Preparation 14) (100 mg, 0.200 mmol) in DCM (1 ml), at room temperature was treated with trifluoroacetic acid (0.5 ml, 6.49 mmol). The resulting mixture was stirred at this temperature for 2 hours then concentrated in vacuo. The residue was loaded onto a SCX column and eluted with MeOH then with a 2N NH₃ solution in MeOH. The ammonia fractions were concentrated in vacuo. The residue was dissolved in DCM (1 ml) and treated with a 4N HCl solution in Et₂O (0.5 ml) to give a pale yellow precipitate. The mixture was concentrated in vacuo. The residue was triturated with Et₂O, filtered off and dried under vacuum (ca 15 mbar) at 60° C. for 16 hours to give 7-(5-{(3-chloro-4-[(1-methylethyl)oxy]phenyl}-1,3,4-thiadiazol-2-yl)-2,3,4,5-tetrahydro-1H-3-benzazepine hydrochloride (69 mgs, 79%) as a yellow solid.

LCMS: retention time 1.08 min; [M+H]⁺=400.2

### EXAMPLE 6

### 2-[(1-Methylethyl)oxy]-[5-(2,3,4,5-tetrahydro-1H-3-benzazepin-7-yl)-1,3,4-thiadiazol-2-yl]benzonitrile trifluoroacetic acid salt

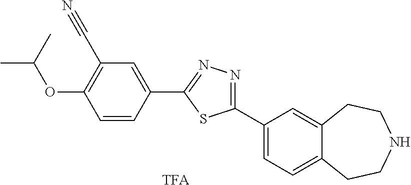

A solution of 1,1-dimethylethyl 7-(5-{3-cyano-4-[(1-methylethyl)oxy]phenyl}-1,3,4-thiadiazol-2-yl)-1,2,4,5-tetrahydro-3H-3-benzazepine-3-carboxylate (Preparation 15) (246 mg, 0.501 mmol) in DCM (5 ml) was treated with trifluoroacetic acid (0.193 ml, 2.507 mmol) and the resulting mixture was stirred at room temperature for 18 hours then concentrated in vacuo. The residue was triturated with Et₂O (5 ml) and filtered off to give 2-[(1-methylethyl)oxy]-[5-(2,3,4,5-tetrahydro-1H-3-benzazepin-7-yl)-1,3,4-thiadiazol-2-yl]benzonitrile trifluoroacetic salt (296 mg, 111%) as a colourless solid.

LCMS: retention time 0.81 min; [M+H]⁺=390.9

### EXAMPLE 6

### Alternative Procedure

### 2-[(1-Methylethyl)oxy]-[5-(2,3,4,5-tetrahydro-1H-3-benzazepin-7-yl)-1,3,4-thiadiazol-2-yl]benzonitrile hydrochloride

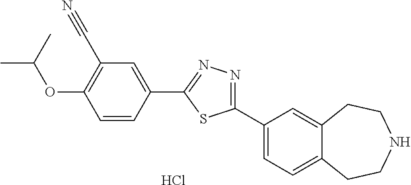

1,1-Dimethylethyl 7-(5-{3-cyano-4-[(1-methylethyl)oxy]phenyl}-1,3,4-thiadiazol-2-yl)-1,2,4,5-tetrahydro-3H-3-benzazepine-3-carboxylate (Preparation 14) (260 mg, 0.530 mmol) in 1,4-dioxane (2 ml) was treated with a 4M HCl solution in 1,4-dioxane (2 ml, 8.00 mmol) and the resulting mixture was stirred at room temperature for 1 hour then concentrated in vacuo. The residue was triturated with AcOEt and the precipitate formed was filtered off and dried under vacuum (ca 15 mbar) to give 2-[(1-methylethyl)oxy]-5-[5-(2,3,4,5-tetrahydro-1H-3-benzazepin-7-yl)-1,3,4-thiadiazol-2-yl]benzonitrile hydrochloride (186 mg, 80%) as a grey solid.

LCMS: retention time 0.81 min; [M+H]⁺=390.9

### EXAMPLE 7

### 4-[7-(5-{3-Cyano-4-[(1-methylethyl)oxy]phenyl}-1,3,4-thiadiazol-2-yl)-1,2,4,5-tetrahydro-3H-3-benzazepin-3-yl]butanoic acid hydrochloride

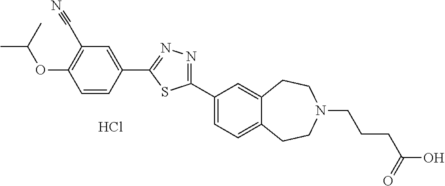

Ethyl 4-[7-(5-{3-cyano-4-[(1-methylethyl)oxy]phenyl}-1,3,4-thiadiazol-2-yl)-1,2,4,5-tetrahydro-3H-3-benzazepin-3-yl]butanoate (Preparation 16) (126 mg, 0.250 mmol) in EtOH (2 ml) at room temperature was treated with a 2N NaOH aqueous solution (0.250 ml, 0.499 mmol). The resulting mixture was stirred at this temperature for 20 hours then treated with acetic acid (0.031 ml, 0.549 mmol) and a 2M HCl aqueous solution (0.5 ml). The resulting solution was concentrated using a freeze-drier to give 4-[7-(5-{3-cyano-4-[(1-methylethyl)oxy]phenyl}-1,3,4-thiadiazol-2-yl)-1,2,4,5-tetrahydro-3H-3-benzazepin-3-yl]butanoic acid hydrochloride (146 mg, 82%) as a straw coloured amorphous solid.

LCMS: retention time 0.92 min; [M+H]⁺=477.3

### EXAMPLE 8

### 2-[(1-Methylethyl)oxy]-[5-(5-methyl-1,2,3,4-tetrahydro-6-isoquinolinyl)-1,3,4-thiadiazol-2-yl]benzonitrile hydrochloride

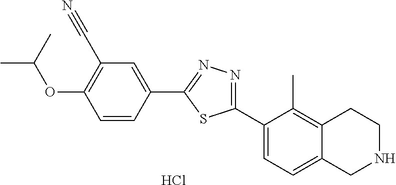

To a solution of 1,1-dimethylethyl 6-(5-{3-cyano-4-[(1-methylethyl)oxy]phenyl}-1,3,4-thiadiazol-2-yl)-5-methyl-3,4-dihydro-2(1H)-isoquinolinecarboxylate (Preparation 21) (173 mg, 0.353 mmol) in 1,4-dioxane (2 ml) at room temperature was added a 4M HCl solution in 1,4-dioxane (2 ml) dropwise and the resulting mixture was stirred at this temperature for 5 hours then concentrated in vacuo. The residue was triturated with Et₂O, filtered off and dried under vacuum (ca 15 mbar) for 16 hours to give 2-[(1-methylethyl)oxy]-[5-(5-methyl-1,2,3,4-tetrahydro-6-isoquinolinyl)-1,3,4-thiadiazol-2-yl]benzonitrile hydrochloride (140 mg, 80%) as a pale yellow solid.

LCMS: retention time 1.16 min; [M+H]⁺=391.15

### EXAMPLE 9

### 3-[6-(5-{3-Cyano-4-[(1-methylethyl)oxy]phenyl}-1,3,4-thiadiazol-2-yl)-5-methyl-3,4-dihydro-2(1H)-isoquinolinyl]propanoic acid sodium salt

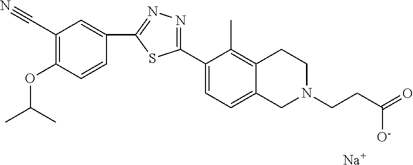

A suspension of ethyl 3-[6-(5-{3-cyano-4-[(1-methylethyl)oxy]phenyl}-1,3,4-thiadiazol-2-yl)-5-methyl-3,4-dihydro-2(1H)-isoquinolinyl]propanoate (Preparation 22) (880 mg, 1.8 mmol) in EtOH (10 ml) was treated with a 2M NaOH aqueous solution (10 ml, 20 mmol). The resulting mixture was stirred heated at 50° C. for 2 hours then cooled to room temperature. Most of the EtOH was concentrated in vacuo. The precipitate formed was filtered off, washed with water and dried under vacuum (ca 15 mbar) to give 3-[6-(5-{3-cyano-4-[(1-methylethyl)oxy]phenyl}-1,3,4-thiadiazol-2-yl)-5-methyl-3,4-dihydro-2(1H)-isoquinolinyl]propanoic acid sodium salt (790 mg, 91%) as a light brown solid.

LCMS: retention time 0.86 min; [M+H]⁺=463.18

### EXAMPLE 10

### 4-[6-(5-{3-Cyano-4-[(1-methylethyl)oxy]phenyl}-1,3,4-thiadiazol-2-yl)-5-methyl-3,4-dihydro-2(1H)-isoquinolinyl]butanoate sodium salt

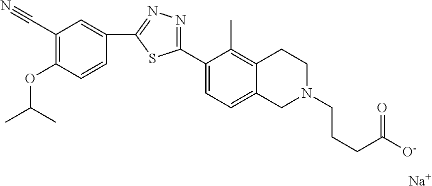

A solution of ethyl 4-[6-(5-{3-cyano-4-[(1-methylethyl)oxy]phenyl}-1,3,4-thiadiazol-2-yl)-5-methyl-3,4-dihydro-2(1H)-isoquinolinyl]butanoate (Preparation 23) (600 mg, 1.19 mmol) in EtOH (3 ml) was treated with a 2M NaOH aqueous solution (5 ml). The resulting mixture was stirred at 50° C. for 1 hour then cooled to room temperature and stirred for another hour. The precipitate formed was filtered off, washed with water and dried to give 4-[6-(5-{3-cyano-4-[(1-methylethyl)oxy]phenyl}-1,3,4-thiadiazol-2-yl)-5-methyl-3,4-dihydro-2(1H)-isoquinolinyl]butanoate sodium salt (438 mg, 74%) as a light brown solid.

LCMS: retention time 0.84 min; [M+H]⁺=476.9

### EXAMPLE 11

### 2-[(1-Methylethyl)oxy]-5-[5-(1,2,3,4-tetrahydro-6-isoquinolinyl)-1,3,4-thiadiazol-2-yl]benzonitrile

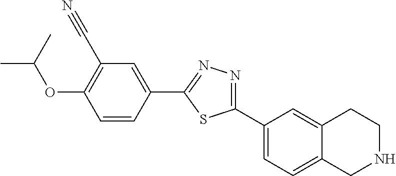

Trifluoroacetic acid (0.5 ml, 6.49 mmol) was added to a solution of 1,1-dimethylethyl 6-(5-{3-cyano-4-[(1-methylethyl)oxy]phenyl}-1,3,4-thiadiazol-2-yl)-3,4-dihydro-2(1H)-isoquinolinecarboxylate (Preparation 26) (380 mg, 0.797 mmol) in DCM (4 ml) at 0° C. under nitrogen. The mixture was allowed to warm to room temperature. After 1 hour, trifluoroacetic acid (0.5 ml, 6.49 mmol) was added and the resulting mixture stirred at this temperature for 3 hours then concentrated in vacuo. The residue was loaded in MeOH onto a SCX cartridge and eluted with MeOH followed by a 2N NH₃ solution in MeOH. The ammonia fractions were concentrated in vacuo to give 2-[(1-methylethyl)oxy]-[5-(1,2,3,4-tetrahydro-6-isoquinolinyl)-1,3,4-thiadiazol-2-yl]benzonitrile (326 mg, 109%) as a pale yellow oil.

LCMS: retention time 0.85 min; [M+H]⁺=377.21

### EXAMPLE 12

### 2-[(1-Methylethyl)oxy]-5-[5-(1,2,3,4-tetrahydro-5-isoquinolinyl)-1,3,4-thiadiazol-2-yl]benzonitrile hydrochloride

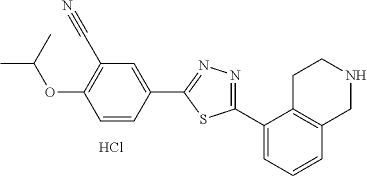

1,1-Dimethylethyl 5-(5-{3-cyano-4-[(1-methylethyl)oxy]phenyl}-1,3,4-thiadiazol-2-yl)-3,4-dihydro-2(1H)-isoquinolinecarboxylate (Preparation 29) (60 mg, 0.126 mmol) was dissolved in 1,4-dioxane (5 ml) and treated at room temperature with a 4M HCl solution in 1,4-dioxane (0.138 ml, 0.554 mmol). The resulting mixture was stirred for 3 hours. Et₂O (15 ml) was added to the reaction mixture and after 10 minutes of stirring, the white precipitate formed was filtered off and dried under vacuum (ca 15 mbar) to give 2-[(1-methylethyl)oxy]-[5-(1,2,3,4-tetrahydro-5-isoquinolinyl)-1,3,4-thiadiazol-2-yl]benzonitrile (28 mg, 54%).

LCMS: retention time 0.86 min; [M+H]⁺=377.02

### EXAMPLE 13

### 5-(5-{3-Chloro-4-[(1-methylethyl)oxy]phenyl}-1,3,4-thiadiazol-2-yl)-2,3-dihydro-1H-isoindole trifluoroacetic acid salt

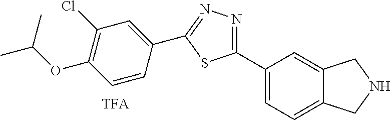

A flask was charged with 1,1-dimethylethyl 5-bromo-1,3-dihydro-2H-isoindole-2-carboxylate (CAS#201940-08-1, commercially available from AB chemical, 53.6 mg, 0.180 mmol), potassium acetate (52.9 mg, 0.540 mmol), 4,4,4′,4′,5,5,5′,5′-octamethyl-2,2′-bi-1,3,2-dioxaborolane (45.7 mg, 0.180 mmol) and 1,1′-bis(diphenylphosphino)ferrocenedichloro palladium(II) (13.16 mg, 0.018 mmol) then filled with DME (2 ml) and the resulting mixture was stirred at 120° C. for 60 minutes under microwave irradiation. 2-Bromo-5-{3-chloro-4-[(1-methylethyl)oxy]phenyl}-1,3,4-thiadiazole (Preparation 11) (60.0 mg, 0.180 mmol) was added along with a saturated NaHCO₃ aqueous solution (0.2 ml). The resulting mixture was stirred at 120° C. for 60 minutes under microwave irradiation then at 140° C. for 30 minutes, still under microwave irradiation. The insoluble material was filtered off via a pad of silica gel and rinsed with DCM and AcOEt. The combined organic phases were concentrated in vacuo and the residue dissolved in DCM (5 ml) and trifluoroacetic acid (3 ml, 38.9 mmol). The resulting mixture was stirred at room temperature for 2 hours then concentrated in vacuo. The residue was loaded on a SCX cartridge and eluted with MeOH then with a 2N NH₃ solution in MeOH. The combined ammonia fractions were combined and concentrated in vacuo. Purification of the residue by MDAP gave 5-(5-{3-chloro-4-[(1-methylethyl)oxy]phenyl}-1,3,4-thiadiazol-2-yl)-2,3-dihydro-1H-isoindole trifluoroacetic acid salt (15.1 mg, 17.3% yield) as a white foam.

LCMS: retention time 1.07 min; [M+H]⁺=372.2

### EXAMPLE 14

### 3-[5-(5-{3-Chloro-4-[(1-methylethyl)oxy]phenyl}-1,3,4-thiadiazol-2-yl)-1,3-dihydro-2H-isoindol-2-yl]propanoic acid

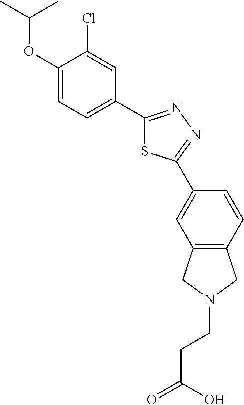

A flask was charged with 1,1-dimethylethyl 5-bromo-1,3-dihydro-2H-isoindole-2-carboxylate (CAS#201940-08-1, commercially available from AB chemical, 78 mg, 0.262 mmol), potassium acetate (79 mg, 0.809 mmol), 4,4,4′,4′,5,5,5′,5′-octamethyl-2,2′-bi-1,3,2-dioxaborolane (68.5 mg, 0.270 mmol) and 1,1′-bis(diphenylphosphino)ferrocenedichloro palladium(II) (19.74 mg, 0.027 mmol) then filled with DME (2 ml). The resulting mixture was stirred at 120° C. for 60 minutes under microwave irradiation. 2-Bromo-5-{3-chloro-4-[(1-methylethyl)oxy]phenyl}-1,3,4-thiadiazole (Preparation 13) (90 mg, 0.270 mmol) was added along with a saturated NaHCO₃ aqueous solution (0.2 ml) and the resulting mixture was stirred at 120° C. for 60 minutes under microwave irradiation. The insoluble material was filtered off via a pad of silica gel and rinsed with AcOEt. The combined organic phases were dried under MgSO₄ and concentrated in vacuo. The residue was dissolved in DCM (5 ml) and treated with trifluoroacetic acid (1 ml, 12.98 mmol). The resulting mixture was stirred for 1 hour at room temperature then concentrated in vacuo. The residue was loaded on a SCX cartridge and eluted with MeOH then with a 2N Ammonia solution in MeOH. The combined ammonia fractions were combined and concentrated in vacuo. The residue was dissolved in n-BuOH (2.0 ml) and treated with tert-butyl acrylate (0.039 ml, 0.270 mmol) and triethylamine (0.075 ml, 0.540 mmol). The resulting mixture was stirred at 100° C. for 1 hour, cooled to room temperature and concentrated in vacuo. The residue was loaded on a SCX cartridge and eluted with MeOH then with a 2N NH₃ solution in MeOH. The combined ammonia fractions were combined and concentrated in vacuo. The residue was dissolved DCM (5 ml) and treated with trifluoroacetic acid (1 ml, 12.98 mmol). The resulting mixture was stirred at room temperature for 2 hours then concentrated in vacuo. Purification of the residue by MDAP gave 3-[5-(5-{3-chloro-4-[(1-methylethyl)oxy]phenyl}-1,3,4-thiadiazol-2-yl)-1,3-dihydro-2H-isoindol-2-yl]propanoic acid (9.8 mg, 8%) as a white foam.

LCMS: retention time 1.06 min; [M+H]⁺=444.2

### EXAMPLE 15

### 3-[6-(2-{3-cyano-4-[(1-methylethyl)oxy]phenyl}-1,3-thiazol-5-yl)-5-ethyl-3,4-dihydro-2(1H)-isoquinolinyl]propanoic acid (E15)

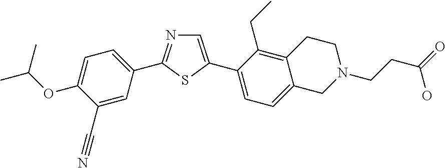

To a solution of 5-[5-(5-ethyl-1,2,3,4-tetrahydro-6-isoquinolinyl)-1,3-thiazol-2-yl]-2-[(1-methylethyl)oxy]benzonitrile (150 mg, 0.372 mmol) and DBU (0.017 mL, 0.112 mmol) in acetonitrile (5 mL) stirred in air was added ethyl 2-propenoate (186 mg, 1.859 mmol) in one portion. The reaction mixture was stirred at 80° C. for 2 hrs. The reaction mixture was evaporated to form a black oil. Lithium hydroxide (25.00 mg, 0.596 mmol) was added to the mixture of the residue obtained, isopropanol (2.5 mL) and water (2.500 mL). The mixture was stirred at rt for 2 hrs and neutralized with AcOH and evaported under high-vacuum, dissolved in THF for MDAP to obtain 3-[6-(2-{3-cyano-4-[(1-methylethyl)oxy]phenyl}-1,3-thiazol-5-yl)-5-ethyl-3,4-dihydro-2(1H)-isoquinolinyl]propanoic acid (89 mg, 50.2% yield).

δH (DMSO, 400 MHz): 12.78 (1H, brs), 8.21 (1H, d), 8.15 (1H, dd), 7.96 (1H, s), 7.37 (1H, d), 7.28 (1H, d), 7.09 (1H, s), 4.86-4.80 (1H, m), 4.51-4.27 (2H, m), 3.74-3.40 (4H, m), 3.08-3.07 (2H, m), 2.82-2.78 (2H, m), 2.63-2.61 (2H, m), 1.29 (6H, d), 1.02 (3H, t) ppm. MS (ES⁺): C₂₇H₂₉N₃O₃S requires 475; found 476 (M+H⁺).

### EXAMPLE 16

### 4-[6-(2-{3-cyano-4-[(1-methylethyl)oxy]phenyl}-1,3-thiazol-5-yl)-5-ethyl-3,4-dihydro-2(1H)-isoquinolinyl]butanoic acid (E16)

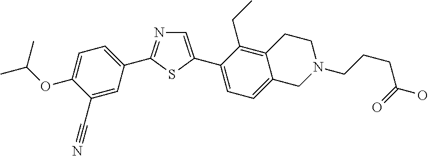

To a solution of 5-[5-(5-ethyl-1,2,3,4-tetrahydro-6-isoquinolinyl)-1,3-thiazol-2-yl]-2-[(1-methylethyl)oxy]benzonitrile (150 mg, 0.372 mmol) and ethyl 4-bromobutanoate (145 mg, 0.743 mmol) in acetonitrile (5 mL) stirred in air was added K₂CO₃ (103 mg, 0.743 mmol) in one portion. The reaction mixture was stirred at reflux overnight. The reaction mixture was evaporated and extracted with EA, washed with water and brine and evaprated. Lithium hydroxide (32.4 mg, 0.773 mmol) was added to the mixture of the residue, isopropanol (2.5 mL) and water (2.500 mL). The mixture was stirred at it for 2 hrs. The reaction mixture was neutralized with AcOH and evaported under high-vacuum, dissolved in THF for MDAP to obtain 4-[6-(2-{3-cyano-4-[(1-methylethyl)oxy]phenyl}-1,3-thiazol-5-yl)-5-ethyl-3,4-dihydro-2(1H)-isoquinolinyl]butanoic acid (82 mg, 34.8 (% yield).

δH (DMSO, 400 MHz): 12.34 (1H, brs), 8.21 (1H, d), 8.15 (1H, dd), 7.96 (1H, s), 7.37 (1H, d), 7.28 (1H, d), 7.12 (1H, s), 4.86-4.80 (1H, m), 4.64-4.50 (1H, m), 4.35-4.30 (1H, m), 3.73 (1H, brs), 3.33-3.27 (1H, m), 3.24-3.19 (21-1, m), 3.14-3.09 (2H, m), 2.63-2.61 (2H, m), 2.35-2.32 (2H, m), 1.94-1.90 (2H, m), 1.29 (6H, d), 1.02 (3H, t) ppm. MS (ES⁺): C₂₈H₃₁H₃O₃S requires 489; found 490 (M+H⁺).

### EXAMPLE 17

### 3-[6-(5-{3-chloro-4-[(1-methylethyl)oxy]phenyl}-1,3,4-thiadiazol-2-yl)-5-ethyl-3,4-dihydro-2(1H)-isoquinolinyl]propanoic acid (E17)

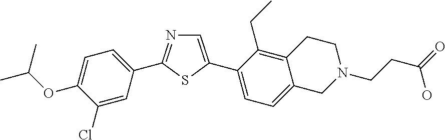

To a solution of 5-[5-(5-ethyl-1,2,3,4-tetrahydro-isoquinolin-6-yl)-thiazol-2-yl]-2-isopropoxybenzonitrile (150 mg, 0.372 mmol) and DBU (0.017 mL, 0.112 mmol) in acetonitrile (5 mL) stirred in air was added ethyl 2-propenoate (186 mg, 1.859 mmol) in one portion. The reaction mixture was stirred at 80° C. for 2 hrs. The reaction mixture was evaporated to form a black oil. Lithium hydroxide (25.00 mg, 0.596 mmol) was added to the mixture of the residue obtained, isopropanol (2.5 mL) and water (2.500 mL). The mixture was stirred at it for 2 hrs and neutralized with AcOH and evaported under high-vacuum, dissolved in THF for MDAP to obtain 3-[6-(5-{3-chloro-4-[(1-methylethyl)oxy]phenyl}-1,3,4-thiadiazol-2-yl)-5-ethyl-3,4-dihydro-2(1H)-isoquinolinyl]propanoic acid (125 mg, 65% yield).

δH (DMSO, 400 MHz): 8.03 (1H, d), 7.89 (1H, dd), 7.48 (1H, d), 7.31 (1H, d), 7.17 (1H, d), 4.80-4.75 (1H, m), 4.44-4.43 (2H, m), 3.39-3.38 (4H, m), 3.10-3.09 (2H, m), 2.81-2.73 (4H, m), 1.28 (6H, d), 1.07 (3H, t) ppm. MS (ES⁺): C₂₅H₂₈ClN₃O₃S requires 485; found 486 (M+H⁺).

### EXAMPLE 18

### 4-[6-(5-{3-chloro-4-[(1-methylethyl)oxy]phenyl}-1,3,4-thiadiazol-2-yl)-5-ethyl-3,4-dihydro-2(1H)-isoquinolinyl]butanoic acid (E18)

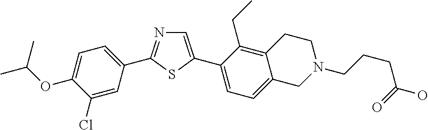

To a solution of 5-[5-(5-ethyl-1,2,3,4-tetrahydro-isoquinolin-6-yl)-thiazol-2-yl]-2-isopropoxybenzonitrile (150 mg, 0.372 mmol) and ethyl 4-bromobutanoate (145 mg, 0.743 mmol) in acetonitrile (5 mL) stirred in air was added K₂CO₃ (103 mg, 0.743 mmol) in one portion. The reaction mixture was stirred at reflux overnight. The reaction mixture was evaporated and extracted with EA, washed with water and brine and evaprated. Lithium hydroxide (32.4 mg, 0.773 mmol) was added to the mixture of the residue, isopropanol (2.5 mL) and water (2.500 mL). The mixture was stirred at it for 2 hrs. The reaction mixture was neutralized with AcOH and evaported under high-vacuum, dissolved in THF for MDAP to obtain 4-[6-(5-{3-chloro-4-[(1-methylethyl)oxy]phenyl}-1,3,4-thiadiazol-2-yl)-5-ethyl-3,4-dihydro-2(1H)-isoquinolinyl]butanoic acid (92 mg, 38% yield).

δH (DMSO, 400 MHz): 12.32 (1H, brs), 8.02 (1H, d), 7.89 (1H, dd), 7.49 (1H, d), 7.31 (1H, d), 7.20 (1H, d), 4.80-4.75 (1H, m), 4.58-4.57 (1H, m), 4.37-4.36 (1H, m), 3.75-3.55 (2H, m), 3.27-3.20 (2H, m), 3.12-3.11 (2H, m), 2.77-2.75 (2H, m), 2.35-2.32 (2H, m), 1.94-1.91 (2H, m), 1.28 (6H, d), 1.07 (3H, t) ppm. MS (ES⁺): C₂₆H₃₀ClN₃O₃S requires 499; found 500 (M+H⁺).

### EXAMPLE 19

### 3-[6-{5-[3-cyano-4-(2-methylpropyl)phenyl]-1,3,4-thiadiazol-2-yl}-5-ethyl-3,4-dihydro-2(1H)-isoquinolinyl]propanoic acid (E19)

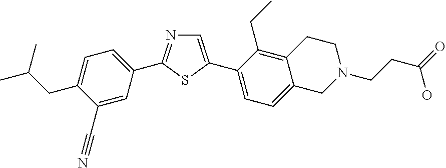

To a solution of 5-[5-(5-ethyl-1,2,3,4-tetrahydro-6-isoquinolinyl)-1,3,4-thiadiazol-2-yl]-2-(2-methylpropyl)benzonitrile (150 mg, 0.372 mmol) and DBU (0.017 mL, 0.112 mmol) in acetonitrile (5 mL) stirred in air was added ethyl 2-propenoate (186 mg, 1.859 mmol) in one portion. The reaction mixture was stirred at 80° C. for 2 hrs. The reaction mixture was evaporated to form a black oil. Lithium hydroxide (25.00 mg, 0.596 mmol) was added to the mixture of the residue obtained, isopropanol (2.5 mL) and water (2.500 mL). The mixture was stirred at rt for 2 hrs and neutralized with AcOH and evaported under high-vacuum, dissolved in THF for MDAP to obtain 3-[6-(2-{3-cyano-4-[(1-methylethyl)oxy]phenyl}-1,3-thiazol-5-yl)-5-ethyl-3,4-dihydro-2(1H)-isoquinolinyl]propanoic acid (93 mg, 52% yield).

δH (DMSO, 400 MHz): 8.39 (1H, d), 8.24 (1H, dd), 7.61 (1H, d), 7.50 (1H, d), 7.19 (1H, d), 4.47-4.46 (2H, m), 3.42-3.39 (2H, m), 3.12-3.11 (2H, m), 2.82-2.77 (4H, m), 2.75-2.70 (2H, m), 1.94-1.91 (1H, m), 1.07 (3H, t), 0.87 (6H, d) ppm. MS (ES⁺): C₂₇H₃₀N₄O₂S requires 474; found 475 (M+H⁺).

### EXAMPLE 20

### 4-[6-(2-{3-cyano-4-[(1-methylethyl)oxy]phenyl}-1,3-thiazol-5-yl)-5-ethyl-3,4-dihydro-2(1H)-isoquinolinyl]butanoic acid (E20)

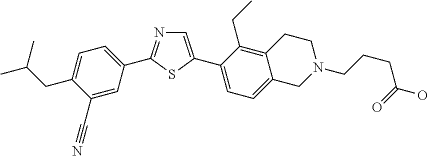

To a solution of 5-[5-(5-ethyl-1,2,3,4-tetrahydro-6-isoquinolinyl)-1,3,4-thiadiazol-2-yl]-2-(2-methylpropyl)benzonitrile (150 mg, 0.372 mmol) and ethyl 4-bromobutanoate (145 mg, 0.743 mmol) in acetonitrile (5 mL) stirred in air was added K₂CO₃ (103 mg, 0.743 mmol) in one portion. The reaction mixture was stirred at reflux overnight. The reaction mixture was evaporated and extracted with EA, washed with water and brine and evaprated. Lithium hydroxide (32.4 mg, 0.773 mmol) was added to the mixture of the residue, isopropanol (2.5 mL) and water (2.500 mL). The mixture was stirred at rt for 2 hrs. The reaction mixture was neutralized with AcOH and evaporated under high-vacuum, dissolved in THF for MDAP to obtain 4-[6-(2-{3-cyano-4-[(1-methylethyl)oxy]phenyl}-1,3-thiazol-5-yl)-5-ethyl-3,4-dihydro-2(1H)-isoquinolinyl]butanoic acid (85 mg, 36% yield).

δH (DMSO, 400 MHz): 12.32 (1H, brs), 8.39 (1H, d), 8.24 (1H, dd), 7.61 (1H, d), 7.51 (1H, d), 7.22 (1H, d), 4.58-4.57 (1H, m), 4.37-4.36 (1H, m), 3.75-3.55 (2H, m), 3.21-3.20 (2H, m), 3.13-3.12 (2H, m), 2.77-2.75 (2H, m), 2.70 (2H, d), 2.35-2.32 (2H, m), 1.96-1.89 (3H, m), 1.08 (3H, t), 0.87 (6H, d) ppm. MS (ES⁺): C₂₈H₃₂N₄O₂S requires 488; found 489 (M+H⁺).

### Membrane Preparation for S1P1 GTPγS Assay

All steps were performed at 4° C. Cells were homogenised within a glass Waring blender for 2 bursts of 15 secs in 200 mls of buffer (50 mM HEPES, 1 mM leupeptin, 25 μg/ml bacitracin, 1 mM EDTA, 1 mM PMSF, 2 μM pepstatin A). The blender was plunged into ice for 5 mins after the first burst and 10-40 mins after the final burst to allow foam to dissipate. The material was then spun at 500 g for 20 mins and the supernatant spun for 36 mins at 48,000 g. The pellet was resuspended in the same buffer as above but without PMSF and pepstatin A. The material was then forced through a 0.6 mm needle, made up to the required volume, (usually x4 the volume of the original cell pellet), aliquoted and stored frozen at −80° C.

### S1P1 GTPγS Assay

S₁P₁ expressing RH7777 membranes (1.5 μg/well) membranes (1.5 μg/well) were homogenised by passing through a 23 G needle. These were then adhered to WGA-coated SPA beads (0.125 mg/well) in assay buffer (HEPES 20 mM, MgCl₂ 10 mM, NaCl 100 mM and pH adjusted to 7.4 using KOH 5M). GDP 10 μM FAC and saponin 90 μg/ml FAC were also added

After 30 minutes precoupling on ice, the bead and membrane suspension was dispensed into white Greiner polypropylene LV 384-well plates (5 μl/well), containing 0.1 μl of compound. 5 μl/well [³⁵S]-GTPγS (0.5 nM for S₁P₁ or 0.3 nM for S₁P₃ final radioligand concentration) made in assay buffer was then added to the plates. The final assay cocktail (10.1 μl) was then sealed, spun on a centrifuge, then read immediately on a Viewlux instrument.

Examples 1 to 14 had a pEC50>5 in this assay.

### S1P1 Tango Assay—384 Well Format

Recombinant EDG1-bla/U2OS cells (contain the human Endothelial Differentiation Gene 1 (EDG1) linked to a TEV protease site and a Ga14-VP16 transcription factor stably integrated into the Tango GPCR-bla U2OS parental cell line) were harvested from growth medium and passaged into assay medium (Invitrogen Freestyle Expression Medium). The cells were starved for 24 hours at 37° C., 5% CO₂, harvested and resuspended in assay medium at a density of ˜200,000 cells/ml. All test compounds were dissolved in DMSO at a concentration of 10 mM and were prepared in 100% DMSO to provide 10 point dose response curves. Test compounds prepared by Bravo (Velocity11) were added to wells in columns 2-11 and 13-22; DMSO was added to wells in columns 12 and 23 as unstimulated controls and assay medium was added to wells in columns 1 and 24 as cell-free controls. An S1P1 agonist was added to wells in row 2, columns 2-11 as stimulated controls and test compounds were added to wells in row 2, columns 13-22 and rows 3-15, columns 2-11/13-22 (row 1 and 16 were empty and not used). Compounds in solution were added to the assay plate (Greiner 781090) using an Echo (Labcyte) dose-response program (50 nl/well). The unstimulated and cell-free controls were loaded with 50 nl/well pure DMSO to ensure that the DMSO concentration was constant across the plate for all assays.

50 μl of the cell suspension was added to each well in columns 2-23 of the plate (˜10,000 cells per well). 50 μl of assay medium was added to each well in the cell-free controls (columns 1 and 24). The cells were incubated overnight at 37° C./5% CO₂.

10 μl of 6× substrate mixture (LiveBLAzer™-FRET B/G substrate (CCF4-AM) Cat #K1096 from Invitrogen, Inc.) was added to each well using Bravo and the plates incubated at room temperature for 2 h in the dark. The plate was finally read on EnVision using one excitation channel (409 nm) and two emission channels (460 nm and 530 nm).

The blue/green emission ratio (460 nm/530 nm) was calculated for each well, by dividing the background-subtracted Blue emission values by the background-subtracted Green emission values. The dose response curve is based on sigmoidal dose-response model. All ratio data was normalized based upon the maximum emission ratio of positive control and minimum emission ratio of negative control (DMSO) on each plate. The intrinsic activity (IA) of each compound would be the normalized percentage of its maximum response after curve fitting.

Examples 15 to 20 had a pEC50≧9 in this assay.

### S1P3 GTPγS Assay

S₁P₃ expressing RBL membranes (1.5 μg/well) were homogenised by passing through a 23 G needle. These were then adhered to WGA-coated SPA beads (0.125 mg/well) in assay buffer (HEPES 20 mM, MgCl₂ 10 mM, NaCl 100 mM and pH adjusted to 7.4 using KOH 5M). GDP 10 μM FAC and saponin 90 μg/ml FAC were also added

After 30 minutes precoupling on ice, the bead and membrane suspension was dispensed into white Greiner polypropylene LV 384-well plates (5 μl/well), containing 0.1 μl of compound. 5 μl/well [³⁵S]-GTPγS (0.5 nM for S₁P₁ or 0.3 nM for S₁P₃ final radioligand concentration) made in assay buffer was then added to the plates. The final assay cocktail (10.1 μl) was then sealed, spun on a centrifuge, then read immediately on a Viewlux instrument.

Examples 1 to 14 had a pEC50<4.5 in this assay.

### S1P3 GeneBlazer Assay

GeneBLAzer EDG3-Ga15-NFAT-bla HEK 293T cells (contain the human Endothelial Differentiation G-protein Coupled Receptor 3 (EDG3) and a beta-lactamase reporter gene under control of a NFAT response element and a promiscuous G Protein, Ga15, stably integrated into the GeneBLAzer Ga15-NFAT-bla HEK 293T cell line) were suspended in assay medium (99% DMEM, 1% Dialyzed FBS, 0.1 mM NEAA, 25 mM HEPES (pH 7.3), 100 U/ml penicillin, 100 μg/ml streptomycin) at a density of 312, 500 cells/ml. Add 100 μl/well of the assay medium to the cell-free control wells (column 12) and 100 μl/well of the cell suspension to the test compound wells (row 2-8, column 1-10), the unstimulated control wells (DMSO) (column 11), and stimulated control wells (S1P) (row 1, column 1-10) in a Corning black-well, clear bottom 96-well plate. Cells were incubated at 37° C., 5% CO2 for 24 h.

Add 25 μl of 5× stock solution of test compounds in assay medium with 0.5% DMSO to the test compound wells, 25 μl of 5× stock solution of agonist (S1P) in assay medium with 0.5% DMSO to the stimulated compound wells, and 25 μl of 5× stock solution of 0.5% DMSO in assay medium to the unstimulated control and cell-free Control wells.

After incubation at 37° C., 5% CO2 for 5 h, 25 μl of 6× substrate mixture (6 μl Solution A (1 mg LiveBLAzer™-FRET B/G Substrate (CCF4-AM) in 912 μl DMSO) plus 60 μl Solution B plus 934 μl Solution C) was added to each well and incubate at room temperature for 2 h in dark. The plate was finally read on EnVision for two emission channels (460 nm and 530 nm).

All test compounds were dissolved in DMSO at a concentration of 10 mM and were prepared in 100% DMSO using a 1 in 5 dilution step to provide 10 point dose response curves. The dilutions were transferred to the assay plates ensuring that the DMSO concentration was constant across the plate for all assays.

Calculate the blue/green emission ratio (460 nm/530 nm) for each well, by dividing the background-subtracted Blue emission values by the background-subtracted green emission values. The dose response curve is based on sigmoidal dose-response model. All ratio data was normalized based upon the maximum emission ratio of positive control (SIP) and minimum emission ratio of negative control (DMSO) on each plate. The intrinsic activity (IA) of each compound would be the normalized percentage of its maximum response after curve fitting.

Examples 15 to 20 had a pEC50<5 in this assay.

1. A compound of formula (I) or a pharmaceutically acceptable salt thereof: 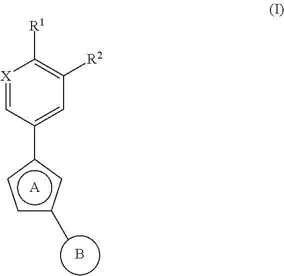
X is CH or N;
R¹ is OR³, NHR⁴, R⁵, NR⁶R⁷, R⁸ or optionally fluorinated C₍₃₋₆₎cycloalkyl;
R² is hydrogen, halogen, cyano, trifluoromethyl, C₍₁₋₂₎alkoxy and C₍₁₋₃₎alkyl optionally substituted by halogen;
R³ and R⁴ are C₍₁₋₅₎alkyl optionally interrupted by 0 and optionally substituted by F or (CH₂)₍₀₋₁₎C₍₃₋₅₎cycloalkyl optionally substituted by F;
R⁵ is C₍₁₋₆₎alkyl optionally substituted by F;
R⁶ and R⁷ are independently selected from C₍₁₋₅₎alkyl optionally interrupted by O and optionally substituted by F and optionally fluorinated C₍₃₋₅₎cycloalkyl with the proviso that the combined number of carbon atoms in R⁶ and R⁷ does not exceed 6;
R⁸ is a 3 to 6 membered, nitrogen-containing heterocyclyl ring optionally substituted by F selected from aziridinyl, azetidinyl, pyrrolidinyl, piperidinyl and morpholinyl, all attached via the nitrogen atom;
A is a 5-membered heterocyclic ring selected from the following: 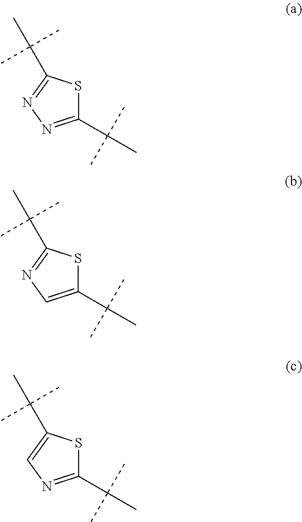
B is a bicyclic ring selected from the following: 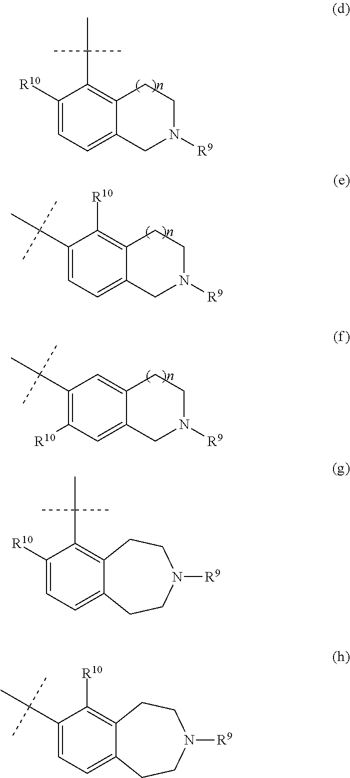
R⁹ is hydrogen or (CH₂)₁₋₄CO₂H;
R¹⁹ is hydrogen or C₍₁₋₃₎alkyl optionally substituted by halogen; and
n is 0, 1 or 2.

2. A compound of formula (I) or a pharmaceutically acceptable salt thereof, wherein:
X is N or CH;
R¹ is OR³ or R⁵;
R³ is isopropyl;
R⁵ is butyl;
R² is chloro or cyano;
A is (a) or (b);
B is (e), (d) or (h);
R⁹ is hydrogen or (CH₂)₂₋₃CO₂H;
R¹⁰ is hydrogen, methyl or ethyl; and
n is 0 or 1

3. A compound according to claim 1 selected from the group consisting of:
7-(2-{3-Chloro-4-[(1-methylethyl)oxy]phenyl}-1,3-thiazol-5-yl)-2,3,4,5-tetrahydro-1H-3-benzazepine;
7-(5-{3-Chloro-4-[(1-methylethyl)oxy]phenyl}-1,3-thiazol-2-yl)-2,3,4,5-tetrahydro-1H-3-benzazepine;
4-[7-(2-{3-Chloro-4-[(1-methylethyl)oxy]phenyl}-1,3-thiazol-5-yl)-1,2,4,5-tetrahydro-3H-3-benzazepin-3-yl]butanoic acid;
4-[7-(5-{3-Chloro-4-[(1-methylethyl)oxy]phenyl}-1,3-thiazol-2-yl)-1,2,4,5-tetrahydro-3H-3-benzazepin-3-yl]butanoic acid;
7-(5-{3-Chloro-4-[(1-methylethyl)oxy]phenyl}-1,3,4-thiadiazol-2-yl)-2,3,4,5-tetrahydro-1H-3-benzazepine;
2-[(1-Methylethyl)oxy]-[5-(2,3,4,5-tetrahydro-1H-3-benzazepin-7-yl)-1,3,4-thiadiazol-2-yl]benzonitrile trifluoroacetic acid;
4-[7-(5-{3-Cyano-4-[(1-methylethyl)oxy]phenyl}-1,3,4-thiadiazol-2-yl)-1,2,4,5-tetrahydro-3H-3-benzazepin-3-yl]butanoic acid hydrochloride;
2-[(1-Methylethyl)oxy]-[5-(5-methyl-1,2,3,4-tetrahydro-6-isoquinolinyl)-1,3,4-thiadiazol-2-yl]benzonitrile;
3-[6-(5-{3-Cyano-4-[(1-methylethyl)oxy]phenyl}-1,3,4-thiadiazol-2-yl)-5-methyl-3,4-dihydro-2(1H)-isoquinolinyl]propanoic acid;
4-[6-(5-{3-Cyano-4-[(1-methylethyl)oxy]phenyl}-1,3,4-thiadiazol-2-yl)-5-methyl-3,4-dihydro-2(1H)-isoquinolinyl]butanoic acid;
2-[(1-Methylethyl)oxy]-[5-(1,2,3,4-tetrahydro-6-isoquinolinyl)-1,3,4-thiadiazol-2-yl]benzonitrile;
2-[(1-Methylethyl)oxy]-[5-(1,2,3,4-tetrahydro-5-isoquinolinyl)-1,3,4-thiadiazol-2-yl]benzonitrile;
5-(5-{3-Chloro-4-[(1-methylethyl)oxy]phenyl}-1,3,4-thiadiazol-2-yl)-2,3-dihydro-1H-isoindole trifluoroacetic acid;
3-[5-(5-{3-Chloro-4-[(1-methylethyl)oxy]phenyl}-1,3,4-thiadiazol-2-yl)-1,3-dihydro-2H-isoindol-2-yl]propanoic acid;
3-[6-(2-{3-cyano-4-[(1-methylethyl)oxy]phenyl}-1,3-thiazol-5-yl)-5-ethyl-3,4-dihydro-2(1H)-isoquinolinyl]propanoic acid;
4-[6-(2-{3-cyano-4-[(1-methylethyl)oxy]phenyl}-1,3-thiazol-5-yl)-5-ethyl-3,4-dihydro-2(1H)-isoquinolinyl]butanoic acid;
3-[6-(5-{3-chloro-4-[(1-methylethyl)oxy]phenyl}-1,3,4-thiadiazol-2-yl)-5-ethyl-3,4-dihydro-2(1H)-isoquinolinyl]propanoic acid;
4-[6-(5-{3-chloro-4-[(1-methylethyl)oxy]phenyl}-1,3,4-thiadiazol-2-yl)-5-ethyl-3,4-dihydro-2(1H)-isoquinolinyl]butanoic acid;
3-[6-{5-[3-cyano-4-(2-methylpropyl)phenyl]-1,3,4-thiadiazol-2-yl}-5-ethyl-3,4-dihydro-2(1H)-isoquinolinyl]propanoic acid; and
4-[6-(2-{3-cyano-4-[(1-methylethyl)oxy]phenyl}-1,3-thiazol-5-yl)-5-ethyl-3,4-dihydro-2(1H)-isoquinolinyl]butanoic acid;
or a salt thereof.

4. A method for treating a condition or disorder mediated by S1P1 receptors comprising administering to a subject with said condition or disorder a therapeutically effective amount of a compound of formula (1) or a pharmaceutically acceptable salt thereof according to claim 1.

5. A method according to claim 4, wherein the condition or disorder is multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, tumour metastasis, diseases associated with angiogenesis, vascular diseases, pain conditions, acute viral diseases, inflammatory bowel conditions, insulin or non-insulin dependant diabetes.

6. A method according to claim 5, wherein the condition is lupus erythematosis.

7-9. (canceled)

10. A pharmaceutical composition comprising a compound according to claim 1.

11-12. (canceled)
